(19) United States
(12) Patent Application Publication
(10) Pub. No.: US 2025/0019740 A1
(43) Pub. Date: Jan. 16, 2025

(54) MUTANT PROTEASES AND USES THEREOF

(71) Applicant: BOARD OF REGENTS, THE UNIVERSITY OF TEXAS SYSTEM, Austin, TX (US)

(72) Inventors: Brent IVERSON, Austin, TX (US); Carl DENARD, Gainesville, FL (US)

(21) Appl. No.: 18/250,645

(22) PCT Filed: Oct. 26, 2021
(86) PCT No.: PCT/US2021/056544

§ 371 Date: Apr. 26, 2023

Related U.S. Application Data
(60) Provisional application No. 63/105,570, filed on Oct. 26, 2020.

(51) Int. Cl. C12Q 1/37; C12N 9/50; C12N 15/10

(52) U.S. Cl. CPC C12Q 1/37; C12N 9/506; C12N 15/1055; C12Y 304/22044

(57) ABSTRACT

The present disclosure, in some aspects, provides mutant TEV proteases that exhibit improved activity, where the mutant TEV exhibits increase efficiency and/or an increased Kcat for cleavage of an amino acid sequence, and TEV proteases are commonly used for laboratory methods including cleaving fusion proteins and removing a purification tag, such as a maltose binding protein or a poly-histidine tag, from a fusion protein or an antibody.

This application is a national phase application under 35 U.S.C. § 371 of International Application No. PCT/US2021/056544, filed Oct. 26, 2021, which claims the benefit of U.S. Provisional Patent Application No. 63/105,570, filed Oct. 26, 2020, the entirety of each of which is incorporated herein by reference.

### BACKGROUND OF THE INVENTION

### 1. Field of the Invention

The present invention relates generally to the field of molecular biology and medicine. More particularly, it concerns mutant proteases and uses thereof.

### 2. Description of Related Art

Proteases are ubiquitous in biology, playing major roles in initiating, regulating, and terminating cellular processes. The diversity and breadth of protease substrate preferences have been harnessed for a variety of applications, including protease therapeutics (Craik et al., 2011), protein purification (Arnau et al., 2006), and mass spectrometry-based proteomics (Giansanti et al., 2016). Proteases are also being utilized for interrogating protein-protein interactions (Kim et al., 2019; Wang et al., 2017), imaging newly synthesized proteins (Lin et al., 2008), and designing synthetic genetic (Sanchez and Ting, 2020; Copeland et al., 2016) and protein circuits (Gao et al., 2018; Fink et al., 2019; Stein and Alexandrov, 2014).

To successfully repurpose proteases for these applications, mutant proteases are typically required. Directed Evolution is one method for engineering proteases. Protease variants with the desired phenotypes are isolated from a large pool, so a high-throughput protease screening system should ideally exhibit a high operational range (sensitivity over a large variation in input), and a high dynamic range (signal-to-noise ratio). Laborious design-build-test cycles slow progress and have been a problem for various screening methods (Sanchez and Ting, 2020; Packer et al., 2017; Pogson et al., 2009; Sellamuthu et al., 2011; Sandersjoo et al., 2014; Li et al., 2013; Varadarajan et al., 2005; Varadarajan et al., 2008a; Varadarajan et al., 2008b).

TEV proteases are used for laboratory and industrial purposes including removing tags from fusion proteins. While some mutant TEV proteases have been generated, additional benefit could be derived from mutant TEV proteases with improved properties or catalytic activity. Clearly, there is a need for improved mutant TEV proteases.

### SUMMARY OF THE INVENTION

In some aspects, the present disclosure overcomes limitations in the prior art by providing mutant TEV proteases that exhibit improved catalytic efficiency, such as an increased k_{cat}. Methods of using the TEV proteases (e.g., for cleavage of fusion proteins, protein purification, etc.) are also provided.

As shown in the below examples, mutant TEV proteases were generated that displayed improved catalytic activity. In select embodiments, a mutant TEV protease (eTEV) was produced that resulted in an 8-fold higher catalytic efficiency compared to the previous TEV mutants (e.g., over TEV-S219P, a mutant TEV protease that displays increased efficiency over the wild-type TEV protease), as well as an increase in turnover rate (k_{cat}) combined with a lowered Kₘ. To the knowledge of the inventors, the data indicates that eTEV is functionally the fastest TEV variant engineered thus far, and eTEV was observed to specifically digest a fusion protein in two hours at a low 1:200 enzyme to substrate ratio. Without wishing to be bound by any theory, structural modeling was consistent with and supports the idea that enhanced interactions between the catalytic Cys151 with the P1 substrate residue (Gln) may contribute to increases in catalytic efficiency of eTEV. As described in the below examples, eTEV routinely outperformed previous mutant TEV proteases (TEV-EAV and TEV-S219P) in fusion protein cleavage assays, demonstrating that eTEV may have particular utility in a variety of methods, including for example as a reagent in protein purification methodologies.

An aspect of the present disclosure relates to a polypeptide comprising an active mutant TEV (Tobacco Etch Virus) protease, wherein the mutant TEV protease comprises: (i) substitution mutations at amino acid residues corresponding to amino acids S3, P8, S31, and A231 of SEQ ID NO:1; and/or (ii) substitution mutation of arginine at amino acid residue corresponding to S219 of SEQ ID NO:1. The mutant TEV protease may further comprises a substitution mutation at amino acid residue corresponding to amino acid T173 of SEQ ID NO: 1. For example, the active mutant TEV protease may comprise alanine at position 173 (T173A). In some embodiments, the mutant TEV protease comprises substitution mutations based on Kabat numbering of: isoleucine at position 3 (S3I), glutamine at position 8 (P8Q), threonine at position 31 (S31T), and a substitution mutation at position 231. The mutant TEV protease may comprise valine at position 231 (A231V). In some embodiments, the mutant TEV protease comprises the substitution mutations of S31, P8Q, S31T, and A231V. In some embodiments, the mutant TEV protease comprises the substitution mutation of S219R. In some embodiments, the mutant TEV protease comprises the substitution mutations of S3I, P8Q, S31T, T173A, and A231V. The mutant TEV protease may further comprise a substitution mutation selected from the group consisting of S219N, S219V, and S219R. In some embodiments, the mutant TEV protease comprises asparagine, valine, or arginine at position 219. In some embodiments, the mutant TEV protease comprises an arginine at position 219. In some embodiments, the mutant TEV protease comprises the substitution mutations of S3I, P8Q, S31T, T173A, S219R, and A231V. The polypeptide may comprise or consist of SEQ ID NO: 3, or a sequence having at least 90%, at least 95%, or at least 99% sequence identity thereto. The polypeptide may comprise or consist of SEQ ID NO:4, or a sequence having at least 90%, at least 95%, or at least 99% sequence identity thereto. In some embodiments, the mutant TEV protease has an amino acid sequence at least 70%, 75%, 80%, 85%, 90%, 95%, or 96% identical to (SEQ ID NO:1). In some embodiments, the mutant TEV protease has an amino acid sequence at least 90%, 95%, or 96% identical to (SEQ ID NO:1), wherein the mutant TEV protease comprises substitution mutations at the amino acid residues corresponding to positions S31, P8Q, S31T, T173A, and A231V of (SEQ ID NO:1). In some embodiments, the mutant TEV protease comprises substitution mutations of S31, P8Q, S31T, T173A, and A231V. The mutant TEV protease may further comprise substitution mutation S219R. In some aspects, the polypeptide can cleave Glu-X-X-Tyr-X-Gln/Xa, (SEQ ID NO:5) at a rate that is faster than the wild-type TEV protease. In some embodiments, the polypeptide can cleave ENLYFQG (SEQ ID NO: 6) or ENLYFQS (SEQ ID NO:7) at a rate of greater than a k_{cat} value of about 0.17 s⁻¹. The k_{cat} value may be at least about or greater than about 0.2, 0.225, 0.25, 0.275, 0.3, 0.325, 0.35, 0.375, or 0.4 s⁻¹. In some embodiments, the k_{cat} value is at least about 0.4 s⁻¹ or is about 0.41 s⁻¹. In some embodiments, the polypeptide can cleave ENLYFQS (SEQ ID NO:7). The polypeptide may further comprises a polypeptide tag sequence. The polypeptide tag sequence may be, for example, a metal binding tag, a histidine tag or polyhistidine tag (His-tag), a HQ tag, a glutathione S-transferase (GST) tag, a N-Utilization substance (NusA) tag, thioredoxin (TRX), trigger factor, SUMO, or a polyarginine tag. In some embodiments, the polypeptide is covalently bound to a maltose binding protein (MBP). In some embodiments, the polypeptide is a fusion protein comprising the maltose binding protein. The polypeptide may be covalently bound to the maltose binding protein or the polypeptide tag sequence via a linker. In some embodiments, the polypeptide comprises a polyhistidine tag or a polyarginine tag. In some embodiments, the polyarginine tag is RRRRR (SEQ ID NO:8). In some embodiments, the substitution mutations are at amino acid residues corresponding to amino acids of (SEQ ID NO: 2). In some embodiments, the polypeptide comprises an N-terminal polyhistidine tag and/or a C-terminal polyarginine tag. The mutant TEV protease may comprise a substation mutation at position 219. In some embodiments, the substitution mutation at position 219 is S219V, S219N, or S219R. In some embodiments, the mutant TEV protease does not comprise a substitution mutation at position 219 or is wild-type at position 219. In some embodiments, at least about 50% of the protease activity remains after storage at 4° C. for 1 week. The polypeptide may be covalently attached to or expressed as a fusion protein with a fluorescent protein. The fluorescent protein may be, e.g., green fluorescent protein (GFP) or superfolder GFP. The fluorescent protein may be attached to the polypeptide via a linker. In some embodiments, the mutant TEV protease comprises or consists of SEQ ID NO:3, SEQ ID NO:4, or SEQ ID NO:18.

Another aspect of the present disclosure relates to a method of cleaving an amino acid bond in a polypeptide comprising contacting the polypeptide with a mutant protease provided above or herein. The polypeptide may comprise the sequence Glu-X-X-Tyr-X-Gln/Xa, (SEQ ID NO:5), wherein the protease cleaves a peptide bond in the sequence. The polypeptide may comprise the sequence ENLYFQG (SEQ ID NO:6) or ENLYFQS (SEQ ID NO: 7). In some embodiments, the polypeptide comprises an antibody variable region. The polypeptide may comprise an antibody. In some embodiments, the polypeptide is further defined as a fusion protein. In some embodiments, the fusion protein comprises a tag, wherein the cleavage separates the tag from the fusion protein. In some embodiments, the tag is a maltose binding protein (MBP), a metal binding tag, a histidine tag (polyhistidine tag or His-tag), a HQ tag, a N-Utilization substance (NusA) tag, thioredoxin (TRX), trigger factor, SUMO, or a glutathione S-transferase (GST) tag. In some embodiments, the tag is a maltose binding protein or a histidine tag. Yet another aspect of the present disclosure relates to use of a mutant protease described herein or above to cleave an amino acid bond in a polypeptide as described above.

Another aspect of the present disclosure relates to a kit comprising a polypeptide comprising an active mutant TEV protease as described herein or above and a suitable container. The kit may further comprise DTT, EDTA, glycerol, a detergent, or a combination thereof. The kit may comprise a resin. In some embodiments, the resin has the capacity to bind a polypeptide tag, such as a His-tag, HQ tag, GST tag, or a HTv7tag.

The disclosure also provides compositions comprising the mutant protease. The composition may include one or more of a buffer such as Tris, HEPES or phosphate butter; a detergent such as Triton present in an amount of about 0.01 to 1%, for example at least 0.05, 0.075, 0.09% or 0.1% and less than about 1%, 0.75%, 0.5%, 0.25% 0.2%, 0.15%; a reducing agent such as dithiothreitol (DTT) present in an about of about 0.1 mM to 25 mM, suitably for example at least 0.1 mM, 0.2 mM, 0.3 mM, 0.4 mM, 0.5 mM or 0.75 mM and less than about 25 mM, 20 mM, 15 mM, 10 mM, 5 mM, 2 mM or 1 mM; a chelating agent such as ethylenediaminetetraacetic acid (EDTA) present in about 0.1 mM to 5 mM, suitably at least about 0.01 mM, 0.02 mM, 0.03 mM, 0.04 mM and less than about 5 mM, 4 mM, 3 mM, 2 mM, 1 mM; glycerol for example 25-75% glycerol (e.g., suitably at least about 25%, 30%>, 35%, 40%>, or 45%), and less than about 75%, 70%, 65%, 60% or 55%; and a salt, such as sodium chloride (suitably in an amount of less than about 500 mM, 400 mM, 300 mM, 250 mM, 200 mM, 150 mM, 125 mM, 100 mM, 50 mM, 25 mM, 20 mM, 15 mM, 10 mM, 5 mM, or 1 mM).

As used herein, “mutant TEV protease” refers to any polypeptide that contains a mutant form of a catalytic domain of the nuclear inclusion an (NIa) protease from Tobacco Etch Virus (TEV) as described herein that is an active protease or can cleave an amino acid sequence. For example, the mutant TEV protease can be utilized as a truncated mutant form (e.g., 27 kDa) comprising or consisting of the catalytic domain of the Nia protease from TEV, e.g., alone or optionally comprised in a fusion protein (e.g., comprising one or more tags). In some embodiments, the mutations provided herein can be included in a mutant form of a full-length (NIa) protease (e.g., 49-50 kDa; UniProtKB-P04517 (POLG_TEV)) from Tobacco Etch Virus, e.g., alone or optionally comprised in a fusion protein (e.g., comprising one or more tags).

The term “homology” refers to a degree of complementarity between two or more sequences. There may be partial homology or complete homology (i.e., identity). The terms “homology” and “identity” in the context of two or more nucleic acids or polypeptide sequences, refer to two or more sequences or subsequences that are the same or have a specified percentage of amino acid residues or nucleotides that are the same when compared and aligned for maximum correspondence over a comparison window or designated region as measured using any number of sequence comparison algorithms or by manual alignment and visual inspection. Methods of alignment of sequence for comparison are well-known in the art. Optimal alignment of sequences for comparison can be conducted by the local homology algorithm of Smith et al, 1981, by the homology alignment algorithm of Needleman and Wunsch, 1970, by the search for similarity method of Pearson and Lipman, 1988, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-G: YESS 2.0: an improved YESS system for the engineering and profiling protease specificity. FIG. 1A, Schematic of the YESS plasmid and process for protease engineering. FIG. 1B, Phenotypes of surface-displayed substrate cassettes after interaction with protease in yeast. FIG. 1C, Rapid assembly of substrate and protease parts in YESS 2.0. In the pYESS2 plasmid, the enzyme is under the control of the β-estradiol titratable promoter (LexA-box)₄-pminCYC). On the substrate side, a GFP expression cassette expressed constitutively in E. coli and flanked by BsmBI restriction sites is placed directly downstream of aga2. A BsmBI-mediated golden gate reaction enables the seamless and rapid assembly of a polypeptide or protein substrate display cassette. The enzyme and substrate cassettes can be added independently or sequentially in the pYESS2 scaffold using BsaI or BsmBI assemblies, respectively. Arrows indicate the directionality of Type IIs restriction sites. FIG. 1D, Construction of the EBY100-Tune strain. FIG. 1E, Breakdown of aga2 substrate cassette to enable assembly of substrate parts using Golden Gate. The overhangs are made unique (two hamming distances between any two overhangs) by taking advantage of the redundancy in DNA sequences that translate into the GS linker between parts, effectively rendering parts only position-dependent (amino acid=SEQ ID NO:11; nucleic acid=SEQ ID NO:12). FIG. 1F, Assembly of a six-part substrate cassette into pYESS2. The order of parts was chosen to be HIS-ENLYFQS-FLAG-ENLYFHS-HA-ERS (SEQ ID NO:10). However, parts can be assembled in any order as long as the correct overhangs are chosen. (SEQ ID NO:13). FIG. 1G, B-estradiol dose-dependent induction of TEV-H activity in the presence or absence of an ERS in the enzyme cassette. To calculate fold activity, the ratio of FLAG/HA signal from each B-E induction concentration is normalized to the FLAG/HA ratio of the control sample (no β-E induction).

FIGS. 2A-D: YESS 2.0 evolution of a highly active TEV variant. FIG. 2A, Two TEV-EAV reporters were built to assess TEV-EAV activity in YESS 2.0. FIG. 2B, Activity of TEV-EAV can be tightly controlled using-E in galactose medium. The legends “+ERS” and “-ERS” refer to the presence or absence of an ER retention sequence on the protease cassette. Both constructs are missing an ERS on the substrate cassette. FIG. 2C, Kinetic characterization of evolved TEV variants. Kinetic characterization of TEV variants on the peptide TENLYFQSGGTRW (SEQ ID NO:14). TEV variant eTEV was obtained by combining mutations from TEV-E2 and TEV-S7 (Table 1). Kinetic parameters for uTEV3, a variant published elsewhere (Sanchez and Ting, 2020), were determined on a peptide substrate by HPLC and compared to published results (in parentheses). FIG. 2D, Digestion of an MBP-TEVcs-GST by TEV variants. Ten μg of MBP-TEVcs-GST was mixed with 0.1 μg of TEV variant in a 10 μL reaction and incubated at 30° C. Reactions were run in duplicates.

FIGS. 3A-B: eTEV outperforms uTEV3 on protein substrates and in YESS 2.0. FIG. 3A, MBP-TEVcs-GST cleavage assay comparing eTEV and uTEV3. FIG. 3B, Comparing the activity of uTEV3, eTEV, and TEV-S219V in YESS 2.0. In these constructs, the protease contains an ERS, while the substrate cassette does not.

FIGS. 4A-B: Structural modeling of TEV variants. FIG. 4A, Compared to TEV-S219P, the SI binding pocket in eTEV is further buried inside the protease. FIG. 4B, Overview of the SI binding pocket of eTEV and TEV-S219P.

FIG. 5: Sorting the error-prone library of TEV-EAV.

FIG. 6: Scan of individual clones induced at 20 nM β-E. Scans containing a yellow star were selected for purification and initial characterization in vitro.

FIGS. 7A-B: FIG. 7A, Initial characterization of TEV variants in vitro on synthetic peptide TENLYFQSTRGGW (SEQ ID NO:15). FIG. 7B, SDS-Page gels of purified TEV variants.

FIGS. 8A-E: FIG. 8A, SDS-Page gel of purified eTEV, TEV-S219P and EAV variants. Protein purity was estimated using an image-line image analysis software. Proteins were found to be >90% pure. FIG. 8B, Biochemical characterization of TEV variants S219P (red), TEV-EAV (blue) and eTEV (green) in vitro on synthetic peptide TENLYFQSTRGGW (SEQ ID NO:15). FIG. 8C, SDS-Page gel of purified TEV-S219V and uTEV3 variants. Protein purity was estimated using an image-line image analysis software. FIG. 8D, Biochemical characterization of uTEV3 on synthetic peptide TENLYFQSTRGGW (SEQ ID NO: 15). FIG. 8E, Biochemical characterization of TEV-S219V on synthetic peptide TENLYFQSTRGGW (SEQ ID NO:15).

FIGS. 9A-D: Representative protein gels of MBP-ENLYFQS-GST (SEQ ID NO: 16) digestion by TEV variants. Percent cleavage for each lane was determined using image analysis using imageJ software. The percent cleavage is listed at the top of each lane. Lane 1 represents a no-enzyme control, showing no cleavage products. In (FIG. 9A) and (FIG. 9B), lanes 2,3,4 and 5,6,7 refer to TEV-S219P, EAV and eTEV, respectively. In this experiment, ten μg of fusion protein was mixed with enzyme in a 10 μL reaction. FIG. 9C, lanes 2, 3, 4 and 6, 7, 8 refer to TEV-S219P, EAV and eTEV, respectively. In this reaction, 5 μg of fusion protein was used in a 20 μL reaction. FIG. 9D, 2, 3, 4 refer to TEV-S219P, EAV and eTEV. In this reaction, 5 μg of fusion protein was used in a 20 μL reaction.

FIG. 10: Representative protein gels of MBP-ENLYFQS-GST (SEQ ID NO: 16) digestion by TEV variants uTEV3 and eTEV. Enzyme to substrate ratio of 1:200 were used for this experiment. The percent cleavage was quantified similarly to FIGS. 9A-D.

FIG. 11: Sorting the error-prone library of TEV-EAV.

FIG. 12: Scan of individual clones induced at 20 nM β-E. Scans containing a yellow star were selected for purification and initial characterization in vitro.

FIGS. 13A-B: FIG. 13A, Initial characterization of TEV variants in vitro on synthetic peptide TENLYFQSTRGGW (SEQ ID NO:15). FIG. 13B, SDS-Page gels of purified TEV variants.

FIGS. 14A-E: FIG. 14A, SDS-Page gel of purified eTEV, TEV-S219P and EAV variants. Protein purity was estimated using an image-line image analysis software. Proteins were found to be >90% pure. FIG. 14B, Biochemical characterization of TEV variants S219P (red), TEV-EAV (blue) and eTEV (green) in vitro on synthetic peptide TENLYFQSTRGGW (SEQ ID NO:15). FIG. 14C, SDS-Page gel of purified TEV-S219V and uTEV3 variants. Protein purity was estimated using an image-line image analysis software. FIG. 14D, Biochemical characterization of uTEV3 on synthetic peptide TENLYFQSTRGGW (SEQ ID NO:15). FIG. 14E, Biochemical characterization of TEV-S219V on synthetic peptide TENLYFQSTRGGW (SEQ ID NO:15).

FIGS. 15A-D: Representative protein gels of MBP-ENLYFQS-GST (SEQ ID NO: 16) digestion by TEV variants. Percent cleavage for each lane was determined using image analysis using imageJ software. The percent cleavage is listed at the top of each lane. Lane 1 represents a no-enzyme control, showing no cleavage products. In (FIG. 15A) and (FIG. 15B), lanes 2,3,4 and 5,6,7 refer to TEV-S219P, EAV and eTEV, respectively. In this experiment, ten μg of fusion protein was mixed with enzyme in a 10 μL reaction. In (FIG. 15C) lanes 2,3,4 and 6,7,8 refer to TEV-S219P, EAV and eTEV, respectively. In this reaction, 5 μg of fusion protein was used in a 20 μL reaction. In (FIG. 15D), 2,3,4 refer to TEV-S219P, EAV and eTEV. In this reaction, 5 μg of fusion protein was used in a 20 μL reaction.

FIG. 16: Representative protein gels of MBP-ENLYFQS-GST (SEQ ID NO: 16) digestion by TEV variants uTEV3 and eTEV. Enzyme to substrate ratio of 1:200 were used for this experiment. The percent cleavage was quantified similarly to FIG. 15.

FIG. 17: Aligned TEV Variants. (WT TEV=SEQ ID NO:17; eTEV=SEQ ID NO: 18; TEV-EAV=SEQ ID NO:19.)

### DESCRIPTION OF ILLUSTRATIVE EMBODIMENTS

### I. Mutant TEV Proteases

The present disclosure, in some aspects, provides mutant TEV proteases that exhibit improved activity. In some embodiments, the mutant TEV exhibits increase efficiency and/or an increased kcat for cleavage of an amino acid sequence such as ENLYFQG (SEQ ID NO: 6) or ENLYFQS (SEQ ID NO:7). TEV proteases are commonly used for laboratory methods including cleaving fusion proteins and removing a purification tag, such as a maltose binding protein or a poly-histidine tag, from a fusion protein or an antibody.

TEV protease typically refers to the 27 kDa catalytic domain of the nuclear inclusion an (NIa) protease from Tobacco Etch Virus (Daros et al., 1999). NIa protein proteases, including TEV protease, recognize a seven amino acid consensus sequence, Glu-X-X-Tyr-X-Gln/Xa, (SEQ ID NO:5) where X can be various amino acid residues, and Xa is serine or glycine. NIa proteases, including TEV protease, normally cleave its substrate between the Gin and Gly/Ser residues. More specifically, TEV protease recognizes a linear epitope of the general form E-Xaa-Xaa-Y-Xaa-Q-(G/S), with cleavage occurring between Q and G or Q and S. The most commonly used sequence is ENLYFQG (SEQ ID NO:6). As compared to other proteases, TEV protease typically displays a high degree to specificity for sequences that are recognized and cleaved. TEV protease typically recognizes the canonical cleavage site, ENLYFQ/G (SEQ ID NO:6) (Parks et al., 1994), and the P1′ position of the substrate may tolerate substitutions with small amino acids (Kapust et al., 2002). The substrate may tolerate different amino acid side chains in the P5, P4 and P2 positions (Xaa) of the substrate, although decreases in processing efficiency may occur due to some substitutions at P2 and P4. TEV protease may often be used at temperature as low as 4° C. to reduce the proteolysis of the target protein. Due to these advantages, TEV protease is frequently used in laboratory methodologies and could be used, for example in the purification process, of therapeutic constructs such as antibodies, immunotoxins, and/or fusion proteins.

Wild-type TEV protease, or full-length wild-type TEV protease, is a 49 kDa NIa protease containing at least two sites at which self-cleavage may occur. The first is at the Vpg cleavage site between the Vpg domain and the catalytic domain which when cleaved yields the Vpg domain of about 22 kDa and the 27 kDa catalytic domain. The second is a self-cleavage site within the 27 kDa catalytic domain, at which cleavage occurs between the methionine at position 218 and the serine residue at position 219 of the catalytic domain. TEV protease is a Cys protease and may be inhibited by thiol reagents such as iodoacetamide.

In certain embodiments, a mutant TEV protease of the disclosure has a specific amino acid sequence identity compared to a wild-type TEV protease (such as, for example, SEQ ID NO:1). In specific embodiments, the mutant TEV protease has an amino acid sequence that is at least 70, 71, 72, 73, 74, 75, 76, 77, 78, 79, 80, 81, 82, 83, 84, 85, 86, 87, 88, 89, 90, 91, 92, 93, 94, 95, 96% or more identical to the amino acid sequence of SEQ ID NO:1. In specific embodiments, the mutant TEV protease has an amino acid sequence that is at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to the amino acid sequence of SEQ ID NO:3. In specific embodiments, the mutant TEV protease has an amino acid sequence that is at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to the amino acid sequence of (GenBank #AZL87748.1). A mutant TEV protease enzyme of the disclosure may be of a certain length, including at least or no more than 239 amino acids in length, for example. The mutant TEV protease may or may not be labeled. The mutant TEV protease may be further modified, such as comprising new functional groups such as phosphate, acetate, amide groups, or methyl groups, for example. The mutant TEV protease may be phosphorylated, glycosylated, lapidated, carbonylated, myristoylated, palmitoylated, isoprenylated, farnesylated, alkylated, hydroxylated, carboxylated, ubiquitinated, deamidated, contain unnatural amino acids by altered genetic codes, and/or contain unnatural amino acids incorporated by engineered synthetase/tRNA pairs, and so forth. The skilled artisan recognizes that post-translational modification of the enzymes may be detected by one or more of a variety of techniques, including at least mass spectrometry, Eastern blotting, Western blotting, or a combination thereof, for example.

The mutant TEV protease may comprise substitution mutations at 1, 2, 3, 4, 5, 6, or all of S3, P8, S31, A231, E79, and/or V219 of SEQ ID NO:1. In some embodiments, the mutant TEV protease may comprise a substitution mutation at (i) amino acid residues corresponding to amino acids S3, P8, S31, and A231 of SEQ ID NO:1; and/or (ii) amino acid residue corresponding to amino acid V219 of SEQ ID NO:1. The mutant TEV protease may further comprise a substitution mutation at amino acid residue corresponding to amino acid T173 of SEQ ID NO:1 (e.g., alanine at position 173; T173A). In some embodiments, the mutant TEV protease comprises: (i) isoleucine at position 3 (S3I), glutamine at position 8 (P8Q), threonine at position 31 (S31T), and a substitution mutation at position 231; and/or (ii) a substitution mutation at position 219. In some embodiments, the mutation at position 219 is S219N, S219V, or S219R. In some preferred embodiments, the mutant TEV protease comprises an arginine at position 219. In some embodiments, the mutant TEV protease comprises mutations S3I, P8Q, S31T, and A231V. In some embodiments, the mutant TEV protease comprises mutation S219R. In some embodiments, the mutant TEV protease comprises the substitution mutations of S31, P8Q, S31T, T173A, and A231V, and preferably a substitution mutation at position S219 (e.g., S219R).

As shown in the below examples, mutant TEV proteases comprising mutations of either (S31, P8Q, S31T, A231V) and/or (V219R) each resulted in improvements in cleavage activity. When these mutations were combined into a single “eTEV” mutant (including S3I, P8Q, S31T, A231V, T173A, and V219R), additional increases in cleavage activity were observed. eTEV displayed a 2-fold improvement in catalytic efficiency (k_{cat}/K_{M}) compared to TEV-EAV, corresponding to a ˜8-fold overall increase in catalytic efficiency relative to the parent TEV-S219P. Notably, while the K_{M} of eTEV remained unchanged at 65 μM with that of TEV-EAV, the turnover rate (k_{cat}) was improved from 0.17 s⁻¹ to 0.41 s⁻¹. Without wishing to be bound by any theory, it is anticipated that since the eTEV has an approximately 2.8-fold lower K_{M} and 2.7-fold higher k_{cat}, the data is consistent with the idea that a buried and rearranged SI binding pocket in eTEV may facilitate a tighter substrate binding. E79G above indicates that this mutation from the parent TEV-EAV (E79) was reverted back to the “WT” sequence G79.

The eTEV mutant protease (S31, P8Q, S31T, T173A, V219R, A231V) has the following amino acid sequence (SEQ ID NO:3): ILFKGQRDYNPISSTICHLTNESDGHTTTLYGIGFGPFIITNKHLFRRNNGTLLVQSLHG VFKVKNTTTLQQHLIDGRDMIIIRMPKDFPPFPQKLKFREPQREERICLVTTNFQTKSM SSMVSDTSCTFPSSDGIFWKHWIQTKDGQCGSPLVSTRDGFIVGIHSASNFANTNNYF TSVPKNFMELLTNQEAQQWVSGWRLNADSVLWGGHKVFMRKPEEPFQPVKEVTQL M. With a polyarginine tag included in the sequence is: it ILFKGQRDYNPISSTICHLTNESDGHTTTLYGIGFGPFIITNKHLFRRNNGTLLVQSLHG VFKVKNTTTLQQHLIDGRDMIIIRMPKDFPPFPQKLKFREPQREERICLVTTNFQTKSM SSMVSDTSCTFPSSDGIFWKHWIQTKDGQCGSPLVSTRDGFIVGIHSASNFANTNNYF TSVPKNFMELLTNQEAQQWVSGWRLNADSVLWGGHKVFMRKPEEPFQPVKEVTQL MRRRRR (SEQ ID NO:4). The eTEV mutant protease may optionally include one, two, or more tags as a fusion protein. If desired, the eTEV mutant protease may be comprised within a mutant form of the NIa protease from Tobacco Etch Virus.

In some embodiments, the mutant TEV protease may comprise one or more additional mutations to further improve the solubility of the protease. The mutant TEV protease may for example include one or more mutations to enhance solubility as described in van den Berg et al., 2006 and/or Cabrita et al., 2007.

A mutant TEV protease as disclosed herein may contain one or more conservative mutations in addition to the specific substitution mutations disclosed herein. Conservative variants of the mutant TEV protease, or its naturally occurring isoforms and homologs, are encompassed by the present invention. Such conservative mutations include mutations that switch one amino acid for another within one of the following groups:
1. Small aliphatic, nonpolar or slightly polar residues: Ala, Ser, Thr, Pro and Gly;
2. Polar, negatively charged residues and their amides: Asp, Asn, Glu and Gin;
3. Polar, positively charged residues: His, Arg and Lys;
4. Large aliphatic, nonpolar residues: Met, Leu, He, Val and Cys; and
5. Aromatic residues: Phe, Tyr and Trp.

The types of substitutions selected may be based on the analysis of the frequencies of amino acid substitutions between homologous proteins of different species developed by Schulz et al. (Principles of Protein Structure, Springer-Verlag, 1978, pp. 14-16), on the analysis of structure-forming potentials developed by Chou and Fasman, 1974, or other such methods reviewed by Schulz et al., 1978, and on the analysis of hydrophobicity patterns in proteins developed by Kyte and Doolittle, 1982. The mutant TEV protease may contain natural and/or synthetic amino acids, if desired.

### II. Tags and Methods of Purification

In some aspects, a polypeptide comprising a mutant TEV protease (e.g., eTEV) or mutant NIa protease as described herein can be covalently bonded to a tag (e.g., a polyhistidine tag), heterologous polypeptide, fusion polypeptide, or carrier polypeptide. In some embodiments, the heterologous polypeptide is a peptide or polypeptide tag that can be used to purify, separate, or isolate the mutant TEV protease from a solution (e.g., from a lysate) by having an affinity for, or binding to, a resin or matrix.

The mutant TEV protease may, for example, comprise a tag at the N-terminus and/or the C-terminus of the polypeptide. In some embodiments, the mutant TEV protease contains one tag. In some embodiments, the mutant TEV protease contains two tags. For example, the mutant TEV protease may comprise (an N-terminal polyhistidine tag and a C-terminal polyarginine tag), or (an N-terminal maltose-binding protein tag and a C-terminal polyarginine tag), (a glutathione S-transferase (GST) tag and a His-tag), etc.

In some embodiments, the tag can be used to attach the mutant TEV protease to a solid support or matrix for removal of the mutant TEV protease, e.g., after the enzymatic activity of the mutant TEV protease. For example, in some embodiments the mutant TEV protease can be used for the removal of an affinity tag from a fusion proteins; if the mutant TEV protease contain a tag (such as a C-terminal polyhistidine tag) the mutant TEV protease can be easily removed after the cleavage reaction by passing the reaction through a Ni-chelating resin.

A variety of tags can be attached to the mutant TEV protease. In some embodiments, the tag is covalently attached to or expressed as a fusion protein to the mutant TEV, e.g., at the C-terminal or N-terminal end of the polypeptide. Optionally, the tag can be attached to the mutant TEV protease via a linker peptide or polypeptide sequence. For example, the tag can be a metal binding tag, a polyhistidine tag (also called a histidine tag or His-tag), a HQ tag, a glutathione S-transferase (GST) tag, N-Utilization substance (NusA), thioredoxin (TRX), trigger factor, SUMO, a polyarginine tag, a maltose binding protein (MBP).

Maltose-binding protein (MBP) can be used as a tag. For example, the mutant TEV protease can be fused to the C terminus of maltose-binding protein (MBP), which can allow for purification due to the MBP's affinity to cross-linked amylose (starch) (e.g., see Guan et al., 1988; Pryor et al., 1997.

N-Utilization substance (NusA) is another tag that can be expressed as a fusion protein (e.g., see Davis et al., 1999). NusA is a native protein from E. coli that can display favorable cytoplasmic solubility characteristics.

Glutathione S-transferase (GST) can be used. GST was derived from the parasitic helminth Schistosoma japonicum, and GST can allow for single-step purification using glutathione-agarose (e.g., Smith et al., Gene. 1988.

Other tags include thioredoxin, trigger factor, and SUMO. Thioredoxin (TRX) may be useful for expression of cytokines in E. coli that are soluble and biologically active (LaVallie et al., 1993). Trigger factor can be used to affect protein degradation in E. coli (Kandror et al., 1995). SUMO can be expressed as a fusion protein in order to enhance expression and solubility in E. coli (Marblestone et al., 2006).

A His-tag is a peptide of histidine residues at either the N or C terminus of a recombinant protein. Typically, a His-tag includes from 4, 5, 6, 7, 8, 9, 10, 11, 12, 13, 14, or more, or more preferably 4-10 consecutive histidine (His) residues. In some preferred embodiments, the His-tag comprises or consists of six histidine residues (also called a hexahistidine tag).

His-tag purification can use the purification technique of immobilized metal affinity chromatography, or IMAC. In this technique, transition metal ions are immobilized on a resin matrix using a chelating agent such as iminodiacetic acid. The most common ion for his-tag purification of a recombinant protein is Ni²⁺, though Co²⁺, Cu²⁺, and Zn²⁺ can also be used. The his-tag has a high affinity for these metal ions and binds strongly to the IMAC column. Most other proteins (e.g., in the lysate after a reaction between a fusion protein and the mutant TEV protease) will typically either not bind to the resin, or will bind only weakly. Inclusion of a his-tag in the mutant TEV protease and IMAC can be used to purify a cleaved fusion protein or recombinant protein directly from a crude lysate. Subsequent purification steps to further purify a recombinant protein or a portion of a cleaved fusion protein can be performed, if desired.

Imidazole can compete with the his-tag for binding to the metal-charged resin and can be used for elution of the mutant TEV protease from an IMAC column. Typically, a low concentration of imidazole can be added to both binding and wash buffers to interfere with the weak binding of other proteins and to elute any proteins that weakly bind. His-tagged protein can then be eluted with a higher concentration of imidazole. Ni²⁺ is most commonly used for his-tag purification since it gives a high yield. Using Co²⁺ can give higher purity but with a lower yield.

In some embodiments, the tag is a metal-affinity tag such as a HQ tag (e.g., a sequence of “HQ” residues that repeat 2, 3, 4, 5, 6, 7, 8, 9, 10 or more times). The tag may comprise or consist of a glutathione S-transferase (GST) tag (position 1 to 654 of Gene ID: 175008). The tag may comprise or consist of a HaloTag® sequence (e.g., as described in Los et al., 2008) or a sequence having at least about 80, 85, 90, 95, 97, 98 or 99% identity with:

(SEQ ID NO: 9)
MAEIGTGFPFDPHYVEVLGERMHYVDVGPRDGTPVLFLHGNPTSSYV
WRNIIPHVAPTHRCIAPDLIGMGKSDKPDLGYFFDDHVRFMDAFIEA
LGLEEVVLVIHDWGSALGFHWAKRNPERVKGIAFMEFIRPIPTWDEW
PEFARETFQAFRTTDVGRKLIIDQNVFIEGTLPMGVVRPLTEVEMDH
YREPFLNPVDREPLWRFPNELPIAGEPANIVALVEEYMDWLHQSPVP
KLLFWGTPGVLIPPAEAARLAKSLPNCKAVDIGPGLNLLQEDNPDLI
GSEIARWLSTLEISG.

Methods of purifying or isolating a protein or polypeptide of interest using a mutant TEV protease as described herein are also provided. For example, the protein or polypeptide of interest can be linked to a polypeptide tag sequence (e.g., a His-tag, a HQ tag, GST tag, or HaloTag®, maltose binding protein (MBP tag), etc.), to form a tagged protein or polypeptide. The polypeptide tag sequence preferably has affinity for a resin or matrix. The tagged protein or polypeptide can contain a cleavage site between the polypeptide tag and the polypeptide or protein of interest which is recognized by the TEV protease (e.g., ENLYFQG (SEQ ID NO:6) or ENLYFQS (SEQ ID NO:7)). The tagged protein or polypeptide can be contacted with the resin or matrix such that it binds to the resin or matrix. Optionally, the protein or polypeptide of interest bound to the resin or matrix is washed one, two, three or more times. The resin or matrix containing the bound tagged protein or polypeptide can then be contacted with the mutant TEV protease which may cleave the protein or polypeptide of interest from the tag, thereby releasing it from the resin or matrix. Subsequent washing of the resin or matrix can be performed to elute the protein or polypeptide of interest. In some embodiments, the protein or polypeptide of interest is an antibody or enzyme.

If desired, both the mutant TEV protease and a fusion protein that can be cleaved by the mutant protease may contain the same tag. In this way, the removal of the mutant TEV protease and the region of the fusion protein (that is released due to cleavage of the fusion protein by the mutant TEV protease) can both be removed in the same purification step. For example, an antibody containing a His-tag may be cleaved by a mutant TEV protease to release the His-tag; if the mutant TEV protease also contains a His-Tag, then the TEV protease and the His-tag that has been separated from the antibody can be removed from a solution or composition containing the antibody in the same purification step.

### III. Fluorescent Proteins

In some embodiments, a polypeptide comprising a mutant TEV protease (e.g., eTEV) as disclosed herein is covalently bound to or expressed as a fusion protein with a fluorescent protein (e.g., GFP, etc.). In some embodiments, inclusion of a fluorescent protein such as superfolder green fluorescent protein (sfGFP) may increase the soluble production of TEV protease.

Variants of sfGFP with different linkers have been generated, for example as described in Wu et al., 2009, and may be covalently bonded to or expressed as a fusion protein with a mutant TEV protease as described herein. It is anticipated that attachment of the sfGFP to the mutant TEV protease may not significantly affect catalytic activity, and may allow for easier detection during expression, purification, or in other methods due to the green fluorescence of the sfGFP.

It is anticipated that a wide variety of fluorescent proteins may be attached to or expressed as a fusion protein with a mutant TEV protease provided herein, For example, other fluorescent proteins that may be attached to the mutant TEV protease include YFP, BFP, CFP, and mVenus.

### IV. Production of TEV Protease

A mutant TEV protease of the present disclosure (e.g., eTEV) can be produced via a variety of methods. In some embodiments, the mutant TEV protease may be produced in a bacterium such as E. coli. In some embodiments, production of the mutant TEV protease is automated.

Expression in E. coli may be desirable for producing mutant TEV proteases that do not require protein refolding. Various mutant TEV protases (e.g., eTEV, or a mutant TEV protease containing the S219R mutation) optionally containing tag(s) as a fusion protein, can be produced in E. coli. The eTEV or a mutant TEV protease containing the S219R mutation may, e.g., be expressed as a fusion protein: (i) containing an N-terminal polyhistidine tag and a C-terminal polyarginine tag, or (ii) containing an N-terminal maltose-binding protein tag and a C-terminal polyarginine tag, and both of these forms can be produced in a high yield in E. coli as soluble proteins that do not require refolding (Kapust et al., 1999, 2001, 2002b).

In some embodiments, chaperone co-expression and low-temperature expression methods may be used, e.g., as described in Fang, 2007. For example, expression of the mutant TEV, optionally expressed as a soluble histidine-tagged TEV, may be achieved using chaperone co-expression and lower temperature fermentation, and the product may be further purified (e.g., using Ni²⁺ affinity chromatography).

The production of the mutant TEV protease may be automated, if desired. For example, in some embodiments an automated two-step purification protocol can be used (e.g., as described in Blommel and Fox, 2007). A fluorogenic substrate can be used to determine the TEV protease's expression and folding in vivo, if desired (Kraft et al., 2007).

The mutant TEV proteases disclosed herein may exhibit improved stability when expressed as compared to the wild-type TEV protease (SEQ ID NO:1). Without wishing to be bound by any theory, it is anticipated that mutation of the S219 position of SEQ ID NO:1 may result in improvements to stability and/or reductions in self-cleavage.

In some embodiments, a mutant TEV protease as described herein may be produced by using expressing the mutant TEV protease as a fusion protein with visible superfolder green fluorescent protein (sfGFP). The sfGFP may be attached to the mutant TEV via a linker in a fusion protein. As described in Wu et al., 2009, purification by Ni-NTA affinity chromatography and Q anion exchange chromatography, using sfGFP-TEV fusion protease, was observed to result in a purity of over 98% and yield of over 320 mg per liter culture.

### V. Kits

The mutant TEV protease may be comprised in a composition in a container means. The container may be included in a kit. The composition may include one or more of a buffer such as Tris, HEPES or phosphate butter; a detergent such as Triton present in an amount of about 0.01 to 1%, for example at least 0.05, 0.075, 0.09% or 0.1% and less than about 1%, 0.75%, 0.5%, 0.25% 0.2%, 0.15%; a reducing agent such as dithiothreitol (DTT) present in an about of about 0.1 mM to 25 mM, suitably at least 0.1 mM, 0.2 mM, 0.3 mM, 0.4 mM, 0.5 mM or 0.75 mM and less than about 25 mM, 20 mM, 15 mM, 10 mM, 5 mM, 2 mM or 1 mM; a chelating agent such as ethylenediaminetetraacetic acid (EDTA) present in about 0.1 mM to 5 mM, suitably at least about 0.01 mM, 0.02 mM, 0.03 mM, 0.04 mM and less than about 5 mM, 4 mM, 3 mM, 2 mM, 1 mM; glycerol for example 25-75%) glycerol, suitably at least about 25%, 30%>, 35%, 40%>, or 45%), and less than about 75%, 70%, 65%, 60% or 55%; and a salt, such as sodium chloride (suitably in an amount of less than about 500 mM, 400 mM, 300 mM, 250 mM, 200 mM, 150 mM, 125 mM, 100 mM, 50 mM, 25 mM, 20 mM, 15 mM, 10 mM, 5 mM, or 1 mM).

The kits generally may comprise, in suitable means, distinct containers for each individual reagent, primer, and/or enzyme. In specific embodiments, the kit further comprises instructions for producing, testing, and/or using enzymes of the disclosure.

### IV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Generation of Mutant TEV Protease with Increased Activity

### YESS 2.0 System and Methodology:

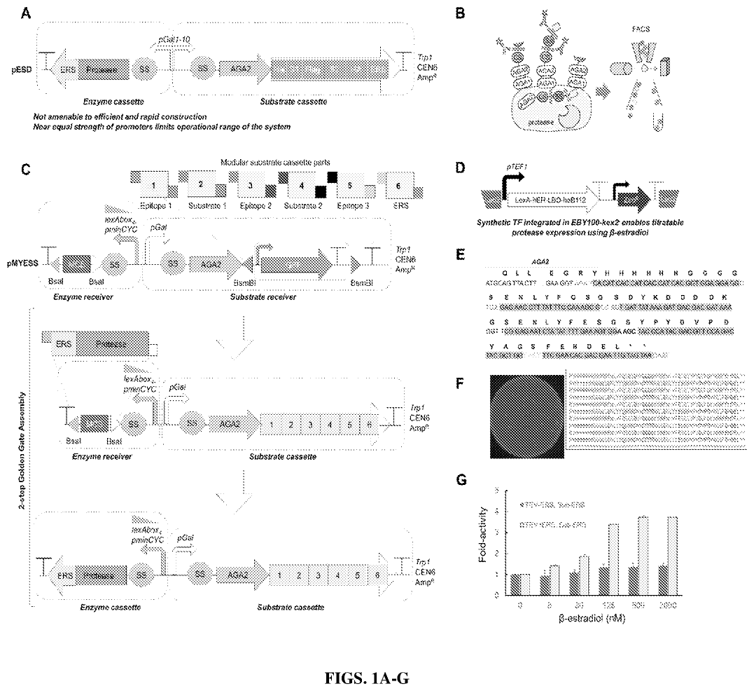

The YESS 2.0 methods used herein utilized two important aspects, including a highly modular vector pYESS2 (FIG. 1C), and an EBY100^{kex2-} (Li et al., 2017) strain with a chromosomally integrated synthetic transcription factor that enables B-estradiol (β-E) induction (Li et al., 2017) (EBY100-Tune) (FIG. 1D). The plasmid, pYESS2, contains four principal features. 1) The transcription of substrate cassette and enzyme are decoupled as they are under the control of β-E and galactose-inducible promoters, respectively. 2) Induction by β-E enables tight control and titration of enzyme levels (and therefore activity), addressing possible protease toxicity and enabling sorting in the absence of enzyme expression (negative sort). 3) Introduction of BsaI and BsmBI sites allow for rapid enzyme and substrate library incorporation via Golden Gate cloning (Engler and Marillonnet, 2014). 4) BsmBI sites on the substrate cassette side flank a constitutively-expressed GFP that drops out upon correct substrate ligation, enabling a rapid initial green-white screen (FIG. 1E) (Lee et al., 2015). The strain EBY100-Tune was generated by integrating the LexA-ER-haB112 synthetic chimeric transcription factor (Cherf and Cochran, 2015) at the leu2 marker of EBY100^{kex2-}. The EBY100^{kex2-} strain is a previously-reported display strain that removes a major yeast endogenous proteolytic background, rendering this platform amenable to profiling proteases with trypsin-like activity (Li et al., 2017).

Previously, the Yeast Endoplasmic Reticulum (ER) Sequestration Screening (YESS) system was developed (Li et al., 2017; Yi et al., 2013; Yi et al., 2015; Taft et al., 2019), which is a robust yeast surface display (YSD) approach that combines for the first time a protease engineering and a comprehensive substrate specificity profiling platform (FIG. 1A). In YESS, an aga2-substrate cassette fusion designed for surface display contains both desired substrates and counter-selection substrates flanked by strategically placed epitope tags. A protease is co-expressed with the substrate cassette, both of which are targeted to the ER of S. cerevisiae. Upon co-expression in the ER, the protease interacts with the transiting substrate fusion, resulting in three possible outcomes: no interaction, cleavage of the selection substrate (SS), or the counter-selection substrate (CS) (FIG. 1B). These phenotypes can be directly quantified by staining the cells with fluorophore-labeled antibodies, allowing visualization and selection by fluorescence-activated cell sorting (FACS). In contrast to modified YSD methods for enzyme engineering (Gai and Wittrup, 2007; Cherf and Cochran, 2015; Konning and Kolmar, 2018; Lim et al., 2017a; Lim et al., 2017b; Mei et al., 2017a), the YESS system (Yi et al., 2013; Yi et al., 2015) marks a significant step in converting YSD, conventionally used to engineer protein binding affinity, thermostability, and solubility into a platform that quantifies catalytic turnover.

Provided herein is YESS 2.0, an improved version of YESS that incorporates features to independently modulate protease and substrate transcription, substrate and protease spatial sequestration within a versatile, seamless, and rapid assembly method (FIG. 1C). Importantly, protease activity can be tuned with significantly higher sensitivity and dynamic range in YESS 2.0 compared to the original YESS platform. Using YESS 2.0, a Tobacco Etch Virus (TEV) variant (eTEV) was engineered with an 8-fold increased catalytic efficiency compared to TEV-S219P. In particular, eTEV shows a 2.73-fold increased catalytic turnover rate (k_{cat}) and a 2.77-fold decreased K_{M}, a noticeable improvement in efficiency under real-use conditions, making it a potentially valuable new tool for protein purification and other applications.

### Rapid Assembly of Protease Activity Reporters:

In YESS 1.0, the substrate cassettes were either assembled using traditional restriction digestion-ligation of a gBlock (aga2-substrate cassette fusion) or by overlap extension PCR. This is because the substrate cassettes downstream of aga2 (substrates, epitope tags, and ERS) are normally as short as possible (<50 bp) to avoid having longer sequences that would increase the chances of containing an inadvertent substrate sequence for a given protease. These obstacles hinder the cloning of parts by traditional cloning or Gibson isothermal assembly. However, with any given enzyme, it is often necessary to 1) test several substrates, 2) permute epitope tags to eliminate adventitious protease substrate sequences and simultaneously optimize surface display, and 3) test various ERS sequences. A multivariable optimization using YESS 1.0, therefore, required many sequential cycles of laborious plasmid construction and analysis.

The second-generation vector pYESS2 was designed to enable the rapid assembly of substrate cassette parts in any desired order, number, and identity as needed. In particular, the substrate cassette sequence downstream of aga2 was divided into short modules connected by a Gly-Ser linker (FIG. 1C). Taking advantage of the degeneracy of the codons that translate into Gly-Ser, unique four base pair (bp) overhangs were designed to ensure that modules ligate in the correct order (FIG. 1E). Therefore, epitope tag and ERS modules can be reused across multiple assemblies. Furthermore, modules are easily assembled by annealing and phosphorylating two DNA oligos in a way that generates the overhangs necessary for ligation. It needs to be pointed out that modules are only position-dependent because their overhangs are within the GS linker and do not interfere each module. The first and last module contain 4-bp overhangs that match the 5′ and 3′-backbone of pYESS2 substrate receiver module. Another strategy used in pYESS2 plasmid is that a GFP gene was originally inserted in the substrate region, which could be replaced by the substrate cassette, thus enabling a white/green selection (FIG. 1C). As an example, the BsmBI assembly efficiency of 6 TEV protease substrate modules in pYESS2 resulted in GFP dropout in over 90% of the colonies (FIG. 1F and Table 1). Moreover, all 10 white clones picked and sequenced were correct. BsmBI assembly of 4, 5 and 6 modules are routinely performed with over 90% accuracy. Through a BsaI assembly, the protease can be added to pYESS2 before or after the substrate cassette construction (FIG. 1C). However, the choice of ERS is encoded in the protease reverse primer sequence rather than as a separate module.

TABLE 1
Sequence of parts used to assemble the
6-piece substrate cassette (SEQ ID NOS: 20-31)

| | |
|---|---|
| 2SS-O1- | 5′-AGATACCACCATCATCACCATCACGGTGGAGGA |
| EP1(His)- | GG-3′ |
| O2 | 3′-TGGTGGTAGTAGTGGTAGTGCCACCTCCTCCGA |
| | GT-5′ |
| 2SS-O2- | 5′-CTCAGAGAACTTTATTTCCAAAGCG-3′ |
| ENLYFQS- | 3′-CTCTTGGAAATAAAGGTTTCGCCGTC-5′ |
| O3 | |
| 2SS-O3- | 5′-GCAGTGATTATAAAGATGACGACGATAAA-3′ |
| EP2- | 3′-ACTAATATTTCTACTGCTGCTATTTCCAA-5′ |
| Flag-O4 | |
| 2SS-O4- | 5′-GGTTCGGAGAATCTATATTTTGAAAGTGG-3′ |
| ENLYFES- | 3′-GCCTCTTAGATATAAAACTTTCACCTTCG-5′ |
| O5 | |
| 2SS-O5- | 5′-AAGCTACCCATACGACGTTCCAGACTACGCTGG-3′ |
| EP3-HA- | 3′-ATGGGTATGCTGCAAGGTCTGATGCGACCATCA-5′ |
| O6 | |
| 2SS-O6- | 5′-TAGTTTCGAACACGACGAATTGTAGTAA-3′ |
| ER-O7 | 3′-AAGCTTGTGCTGCTTAACATCATTGTTA-5′ |

### Modulating Protease Activity in YESS 2.0:

In YESS 1.0, the inability to finely control or turn off protease expression sometimes resulted in unwanted consequences. Since protease activity on a substrate within YESS is typically observed through a loss of fluorescence, mutations or stop codons accumulated within the C-terminal epitope tag or the substrate itself can be enriched by FACS and overtake the sorted population. This phenomenon makes it challenging to assess whether a sorted library contains improved variants or false positives, as a significant fraction of the sorted library may contain epitope tag mutations and stop codons. False positives can only be removed by re-cloning a sorted protease library in a new pESD plasmid. In contrast, the YESS 2.0 system overcomes these obstacles by shifting protease expression to a titratable, orthogonal β-E inducible promoter and enabling one to switch off protease activity independently of substrate expression for use in negative screening rounds.

These YESS 2.0 features were first showcased by building and transforming two TEV-H activity reporters into EBY100-Tune. We had previously identified TEV-H as an orthogonal TEV variant, preferring a P1 His over the wild-type preferred P1 Gln (FIG. 1E). The two reporters constructed differed by the presence or absence of the FEHDEL ERS from the protease cassette, with no ERS present on the substrate end. When the FEHDEL ERS is present, TEV-H activity increases in a dose-dependent manner over nearly three orders of magnitude of β-E concentration (FIG. 1G). In contrast, with the added stringency of an absence of any ERS, TEV-H shows observed activity only the β-E concentration is above 125 nM. This key result verified that, indeed, YESS 2.0 can control enzyme activity at both transcriptional and post-translational levels. Importantly, no protease activity is observed in the absence of β-E.

### Directed Evolution of a Highly Active TEV Protease:

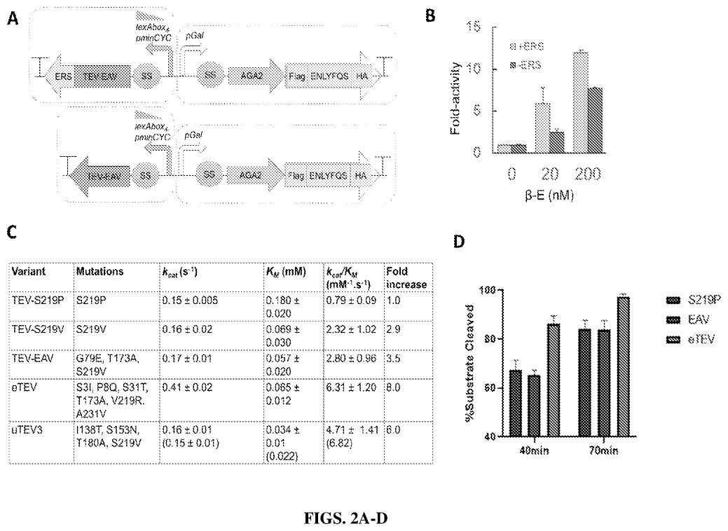

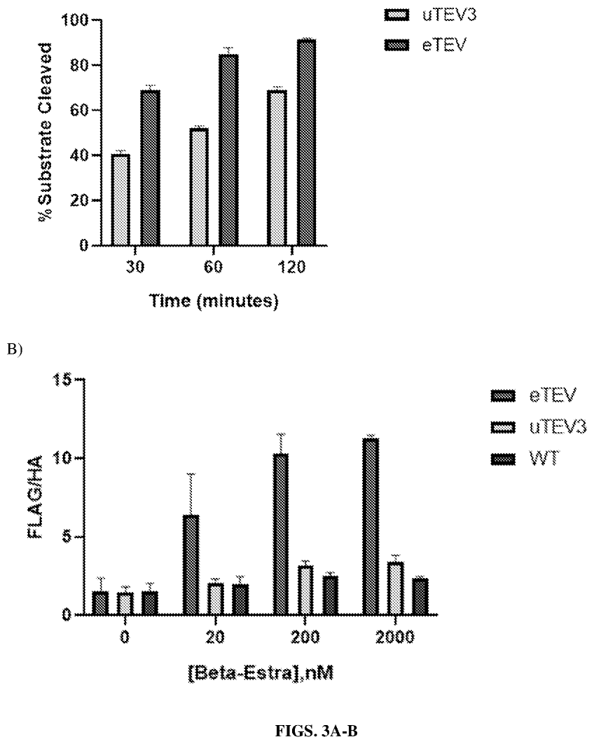

YESS 1.0 was used to evolve a triple mutant TEV variant (G79E, T173A, S219V) (referred to here as TEV-EAV) with 3.5-fold higher catalytic efficiency compared to the parent TEV-S219P when reacting with the native substrate ENLYFQS (SEQ ID NO:7) (Yi et al., 2013). To determine whether further enhanced TEV variants could be isolated using YESS 2.0, TEV-EAV reporter strains were induced at three concentrations of β-E (0, 20, and 200 nM). Induction of a TEV-EAV reporter devoid of ERS with 20 and 200 nM β-E showed a respective 2.5-fold and 7.5-fold increase in activity compared to cells induced with galactose only (FIG. 2A). This assay indicated that inducing a TEV-EAV library with 20 nM β-E or lower would allow variants with higher catalytic efficiency to be selected.

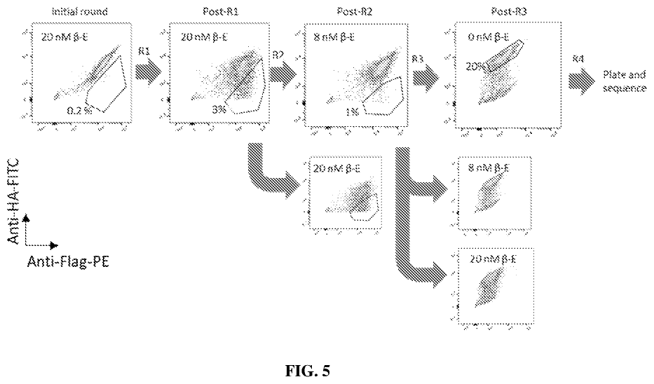

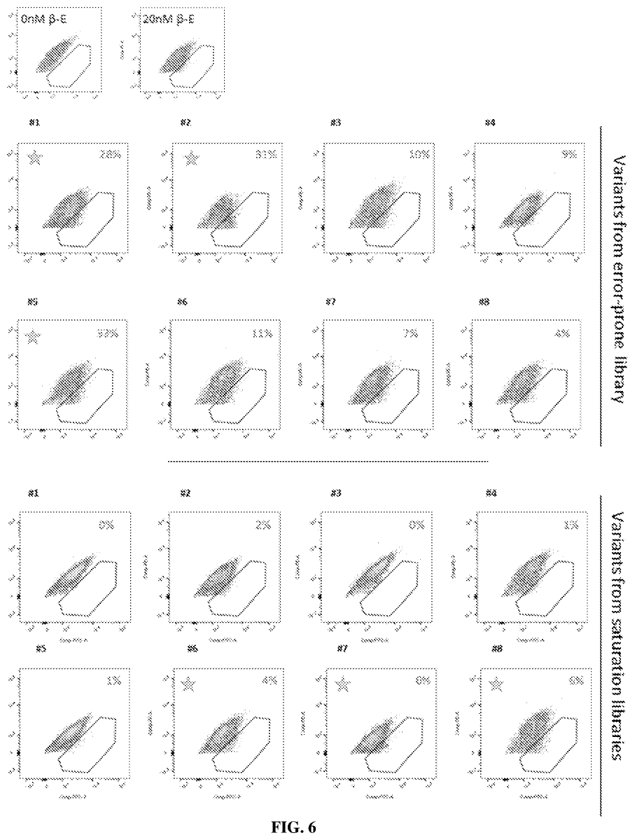

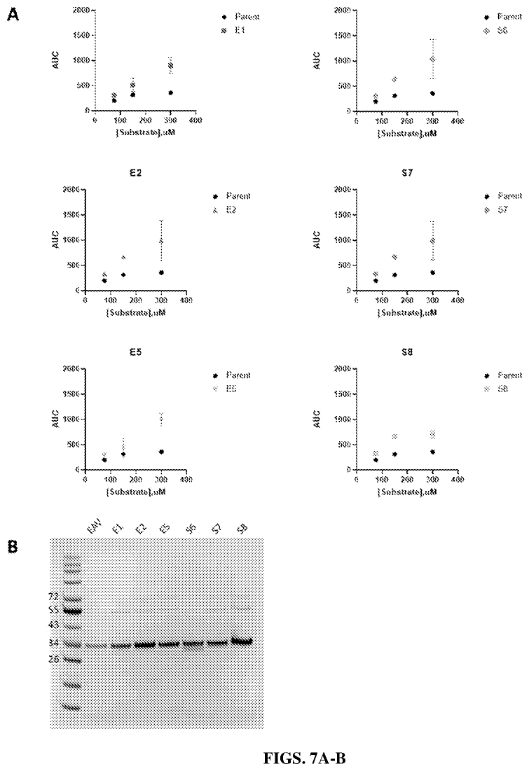

Based on this result, an error-prone (1.5% error rate) and a site-saturation mutagenesis library (at residues E79, A173 and V219) with no ERS on either substrate or protease ends were prepared on the TEV-EAV template and screened against the AGA2-FLAG-ENLYFQS-HA (SEQ ID NO:32) substrate cassette. After being transformed into EBY100-Tune (Benatuil et al., 2010), 2×108 cells were induced in YNB-CAA-galactose supplemented with 20 nM β-E and subsequently labeled with fluorescently-labeled anti-FLAG-PE and anti-HA-FITC antibodies. Cells that displayed high PE and low FITC fluorescence (0.2% of the population) were collected. Post-RI cells were induced with 20 nM and 8 nM β-E in YNB-CAA-galactose, respectively. After analyzing the activity of the library, cells induced with 8 nM β-E that displayed high PE and low HA fluorescence were collected (1% of the population). Finally, a negative sorting round (in the absence of β-E) was performed, and cells displaying both high PE and FITC signals were collected (FIG. 5). This sorting strategy appraised the functionality of the sorted library after each round and can inform at sorting stringency or whether to perform a negative round of sorting. A negative sort allows one to collect cells that display both FLAG and HA epitope tags, effectively removing stop codons and mutations from the library. After four rounds of sorting, all screened clones showed improved cleavage activity in YESS 2.0 compared to parent TEV-EAV (FIG. 6). These phenotypes were retained in vitro as six selected clones showed higher activity on the peptide TENLYFQSGTRRW (SEQ ID NO:33) compared to TEV-EAV (Table 2, FIGS. 7A-B).

TABLE 2
Sequence of selected TEV variants after sorting

| Variant | Mutations |
|---|---|
| E1 | Q74L, S200N |
| | F139(C→T), V216 (T→C), P227(A→T) |
| E2 | S3I, P8Q, S31T, A231V |
| | P95 (T→C), C110 (T→C), N171 (T→C) |
| E5 | K6M, S15L |
| S6 | E79S, A173G, V219S |
| S7 | E79G, S219R |
| S8 | A173D, V219K |

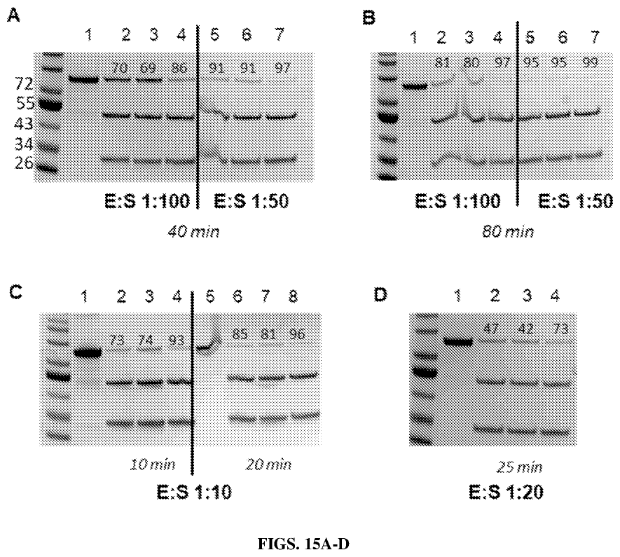

Next, we combined the two best variants, E2 (S3I, P8Q, S31T, A231V) and S7 (E79G, S219R), from the error-prone and saturation mutagenesis libraries, respectively, to generate the hexamutant enhanced TEV “eTEV” (S3I, P8Q, S31T, E79G, T173A, S219R, A231V). This enhanced TEV shows a 2-fold improvement in catalytic efficiency (k_{cat}/K_{M}) compared to TEV-EAV, corresponding to a ˜8-fold overall increase in catalytic efficiency relative to the parent TEV-S219P. Notably, while K_{M} of eTEV remained unchanged at 65 μM with that of TEV-EAV, the turnover rate (k_{cat}) was improved from 0.17 s⁻¹ to 0.41 s⁻¹ (FIG. 2C, FIGS. 14A-E).

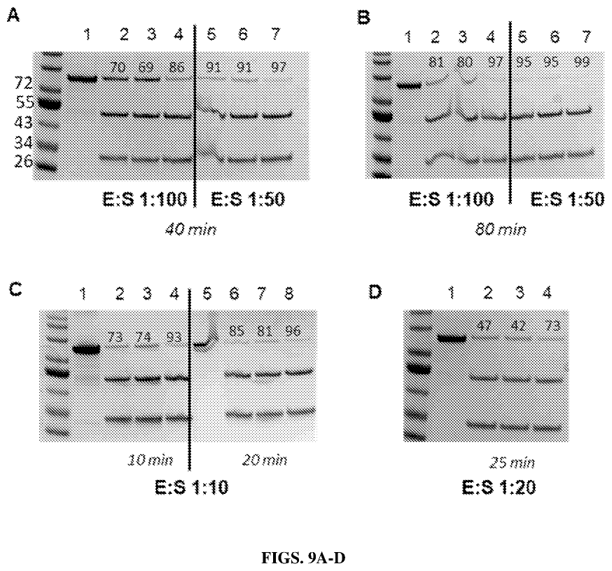

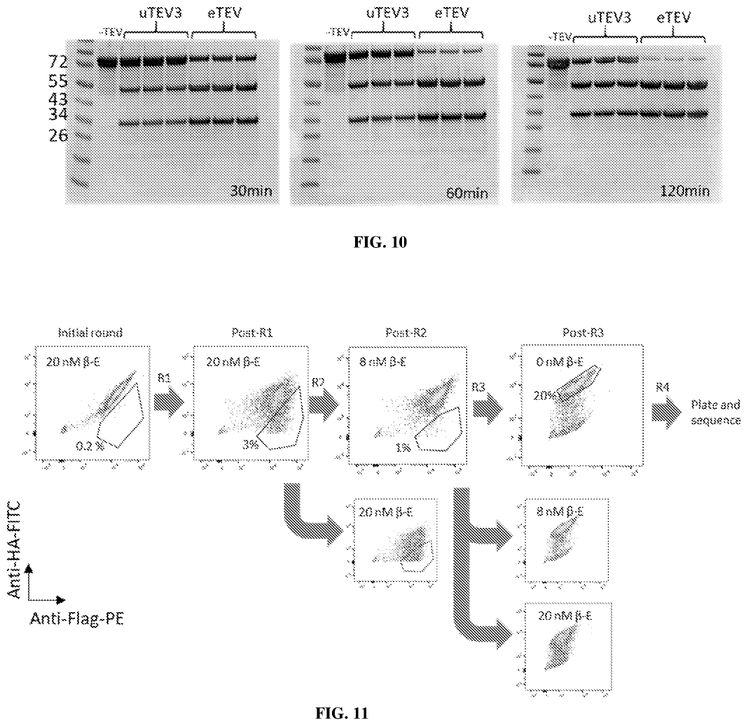

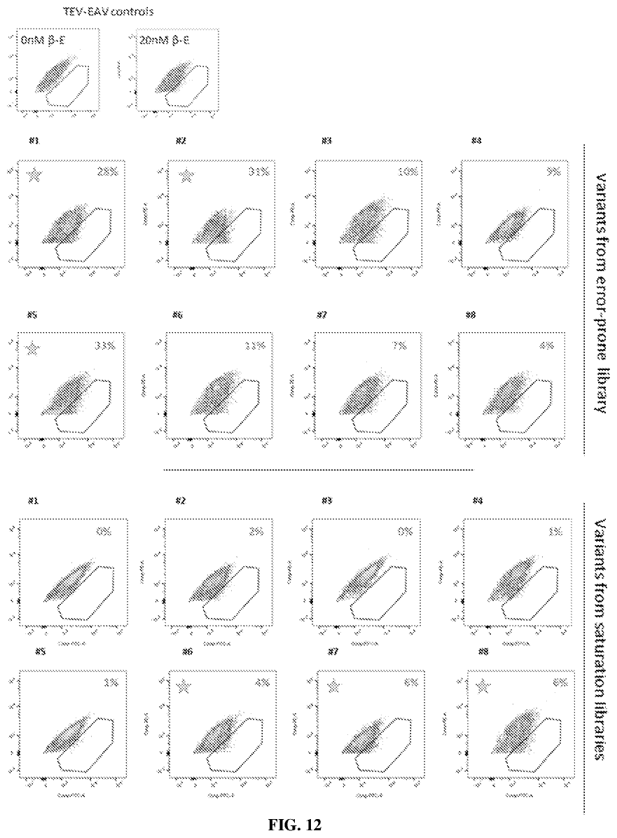

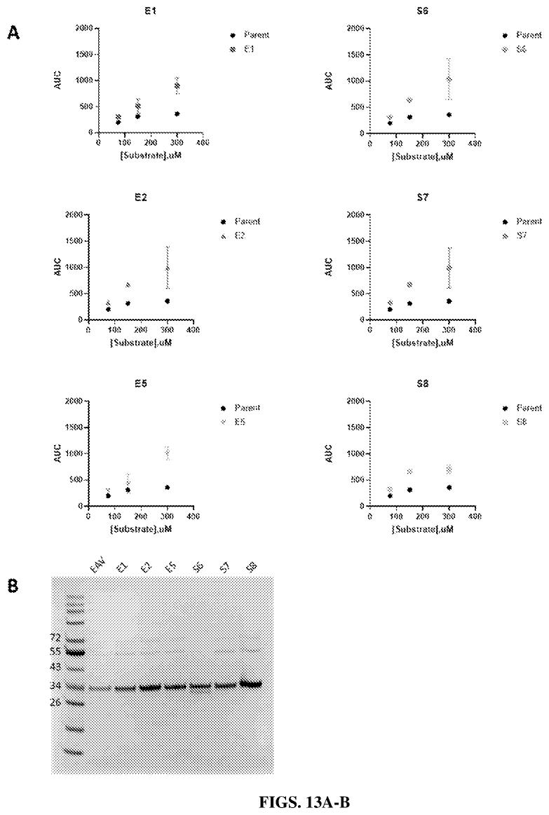

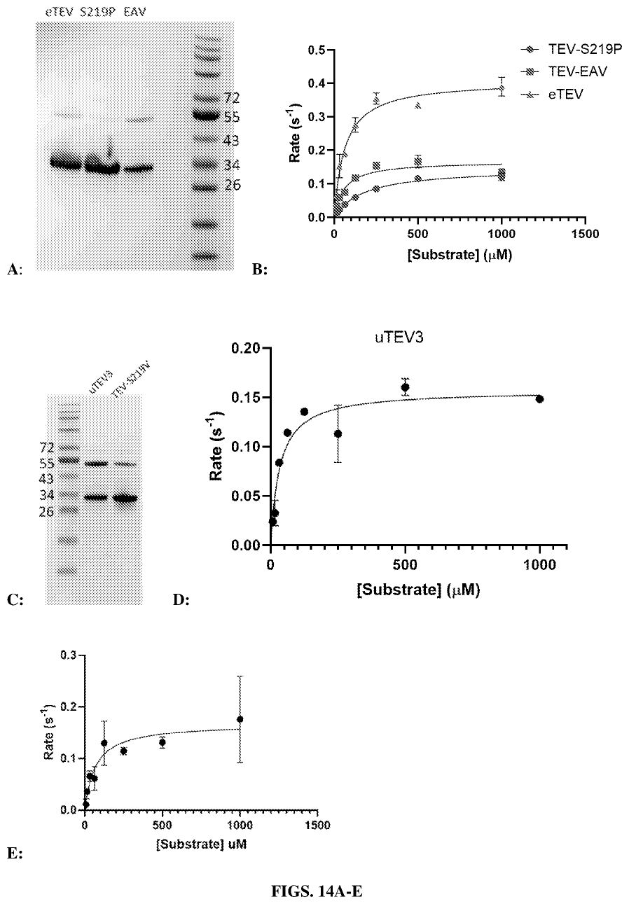

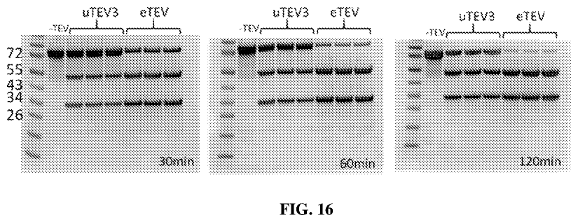

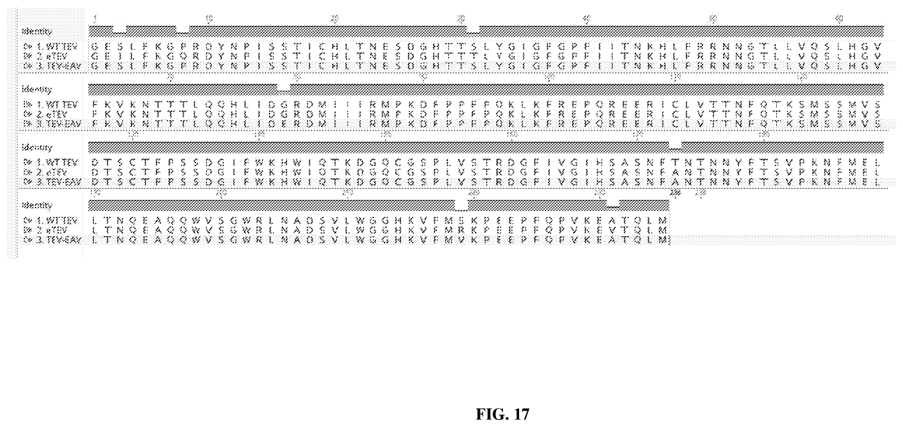

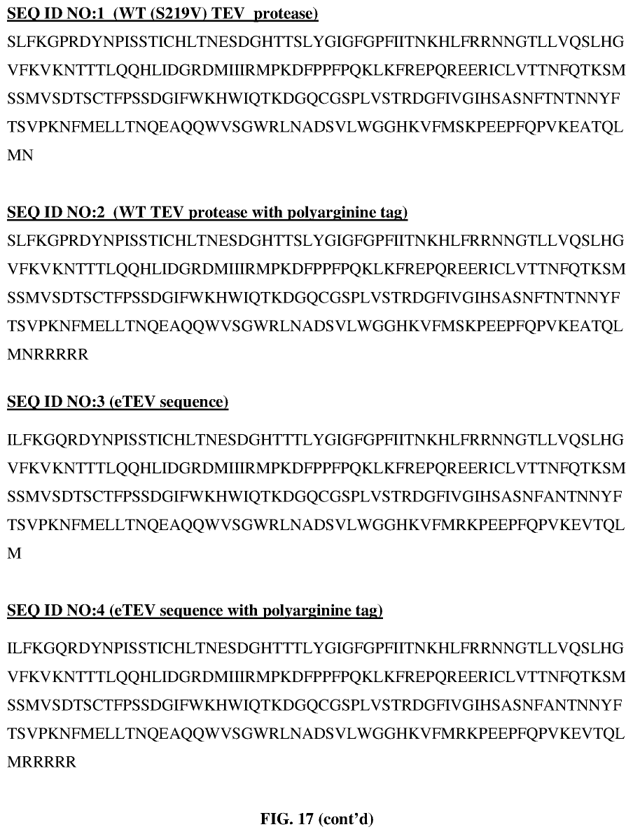

Finally, eTEV was tested in MBP-ENLYFQS-GST (SEQ ID NO:16) fusion protein cleavage assays using conditions recommended for commercial TEV protease. eTEV routinely outperformed TEV-S219P and TEV-EAV at various ratios of enzyme to substrate (FIG. 2D and FIGS. 9A-D). For instance, eTEV was able to digest 85% of the MBP-ENLYFQS-GST (SEQ ID NO: 16) fusion protein in 40 minutes at 30° C. at a 1:100 ratio, compared to ˜68% for TEV-EAV and TEV-S219P (FIG. 2D, FIGS. 15A-D). Therefore, the low amount of eTEV and reduced time needed to reach complete cleavage of a protein fusion (˜70 mins) makes eTEV a valuable reagent for protein purification.

Comparison of eTEV and uTEV3:

uTEV3 was generated using a yeast platform based on TEV-mediated transcription factor release via induction of protein-protein interactions (Sanchez and Ting, 2020). Based on characterization, eTEV and uTEV3 possess similar catalytic efficiencies under the same conditions (FIG. 2C), except that the improved catalytic efficiency in uTEV3 appears to be mainly due to a significant K_{M} decrease. However, in the MBP-ENLYFQS-GST (SEQ ID NO:16) fusion protein cleavage assay, eTEV outperformed uTEV3 at every point along the assay time course (FIG. 3A). After 30 mins, at a 1:200 protease to substrate ratio, eTEV cleaved 70% of the MBP-TEVcs-GST fusion, compared to only 30% cleavage with uTEV3. Furthermore, at a 1:200 ratio, eTEV reached ˜90% digestion of the fusion protein after 120 min (FIG. 16).

As evidence that the TEV engineering strategy in YESS 2.0 was operating at saturating substrate levels, uTEV3 was cloned in a pYESS2 construct with no ERS similar to TEV-EAV and tested by FACS. Under these conditions, uTEV3 showed comparable activity to TEV-S219P, highlighting that our YESS 2.0 approach did not select for TEV variants with stronger substrate-binding interactions.

### Structural Analysis of TEV Variants:

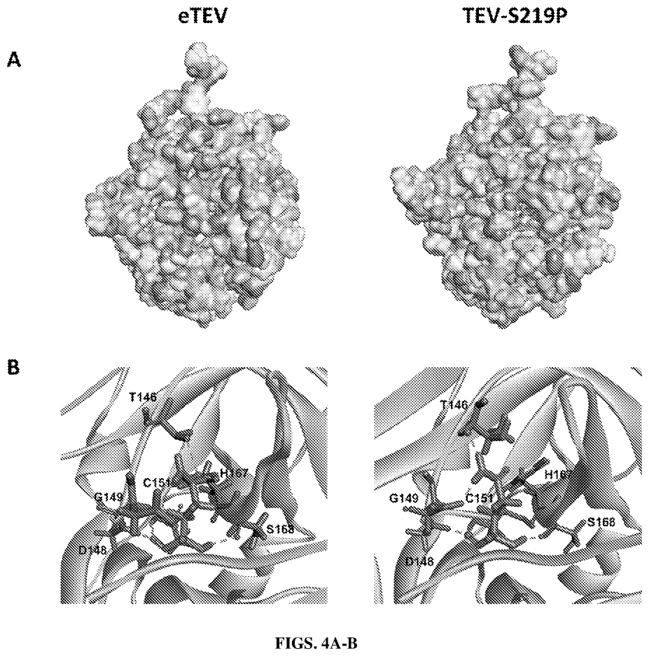

Although eTEV contains 6 mutations, none of them exists in the SI binding pocket or catalytic score. To help understand the better catalytic properties of eTEV, structure modeling of TEV-S219P and eTEV was performed with the peptide substrate, ENLYFQS (SEQ ID NO:7). The results showed that there is a clear substrate channel in the TEV protease for substrate binding. Compared to TEV-S219P, the S1 binding pocket in eTEV was further buried inside the protease (FIG. 4A and FIG. 4B). Without wishing to be bound by any theory, detailed analysis of the SI binding pocket indicates that although the main residues interacting with substrate P1 residue, Gln, in the S1 binding pocket remained the same, the residue interaction in eTEV is tighter, which might be due to the buried and small SI binding pocket (FIG. 4A). Considering that the eTEV has an approximately 2.8-fold lower K_{M} and 2.7-fold higher k_{cat}, the buried and rearranged SI binding pocket in eTEV might facilitate a tighter substrate binding, which prompted its substrate proteolysis.

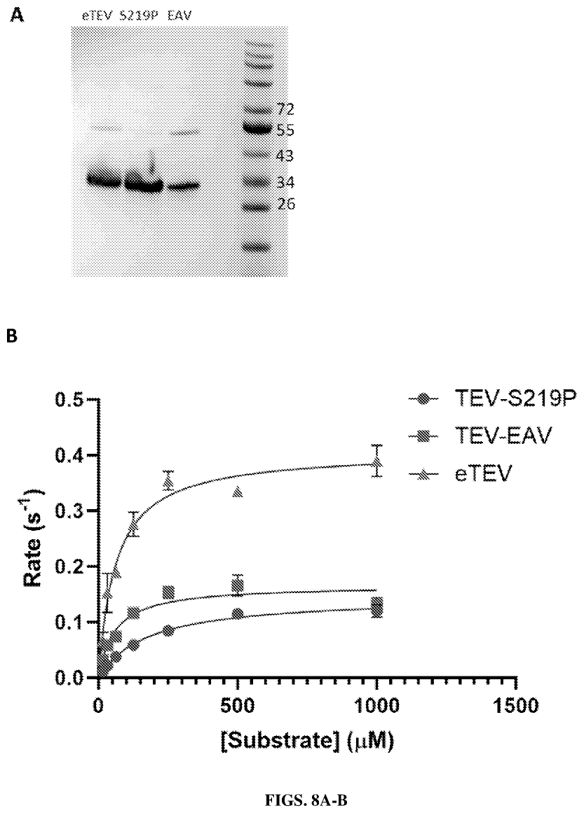

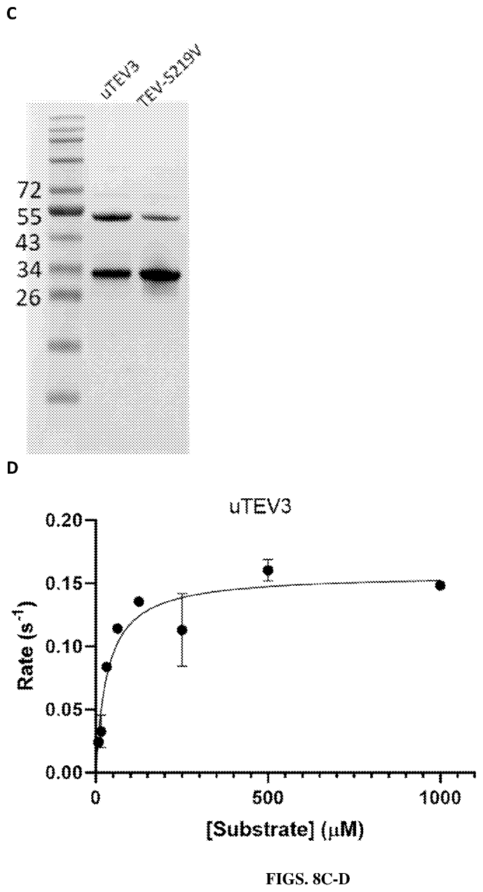

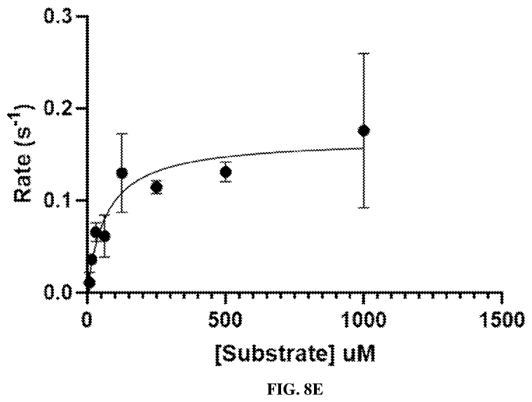

Isolated TEV protease clones from the saturation mutagenesis library were observed to contain variations at position 219 (Table 2). Residue 219 is believed to be part of the flexible C-terminal loop positioned below the catalytic core and plays a critical role in regulating TEV autolysis and substrate binding (2001; Nam et al., 2020). Variant S6 reverted to the wild-type sequence and showed autolysis after purification as expected (FIG. 8B). Importantly, the respective mutations of S219 to Arg and Lys in variants S6 and S7 are shown for the first time to increase the catalytic efficiency of TEV while preventing autolysis (FIG. 7A). Without wishing to be bound by any theory, the S219R mutation in variant S7 supports the idea that it may be responsible for the observed improvements in catalytic efficiency. On the one hand, S219P and S219E prevent TEV autolysis, but occurs at the detriment of k_{cat} and K_{M}, respectively (Kapust et al., 2001). On the other hand, mutating S219 to Val, Asn, Lys, or Arg also prevents autolysis, yet, in addition, they result in increased catalytic activity (Kapust et al., 2001; Nam et al., 2020) (FIG. 2B). Whether residue 219 has greater impacts on K_{M} or k_{cat} may not be straightforward, and may depend on other existing mutations in TEV.

Extensive re-screening of individual clones to confirm activity is an underreported bottleneck of directed evolution experiments. It is therefore noteworthy that 15 out of 16 TEV variants isolated from our screen showed higher activity when analyzed by FACS as an individual clone in YESS 2.0 as well as when the same clone was analyzed using in vitro cleavage assays using a peptide substrate (FIG. 6), thereby circumventing the re-screening bottleneck. Furthermore, by targeting the protease to the ER, the YESS 2.0 system promotes protein folding and could potentially synergize with ER engineering approaches that enhance protein folding and secretion (Mei et al., 2019; de Ruijter et al., 2016; Huang et al., 2018. Such tools might be necessary to facilitate rapid activity tests for uncharacterized and difficult-to-express proteases. Nevertheless, in soon to be reported studies, several different mammalian proteases have now been shown to be active by ER sequestration, including serine (Guerrero et al., 2016) and metalloproteases (Mei et al., 2017b).

Overall, the YESS 2.0 system further improves the YSD approach for understanding and redesigning the specificity of protein-modifying enzymes such as proteases. First, the speed and versatility with which protease activity reporters are constructed and verified in YESS 2.0 compared to YESS 1.0 further improve experimental workflow. Second, the transcriptional and translational control nodes introduced in YESS 2.0 provide for significantly higher sensitivity and dynamic range compared to YESS 1.0. These features allow the system to operate at, or near, saturation kinetics so that TEV variants with higher k_{cat} were selected. To the knowledge of the inventors, while TEV protease has been extensively engineered, eTEV is the first TEV variant with increased k_{cat}.

### Example 2

### Materials and Methods

Construction of pYESS2:

The plasmid pYESS2 was based on the pESD backbone previously described (Yi et al., 2013) with the following modifications. First, the BsaI and BsmBI sites found on the pESD backbone were removed by Gibson Isothermal assembly. Second, an intermediate entry vector was constructed by replacing the substrate and enzyme cassettes of pESD with a gene block containing multiple type II restriction sites. These restriction sites were used to add sequentially a Gall promoter, a β-estradiol-inducible promoter (lexA(box)₄-pCYC1), aga2, a constitutively-expressed GFP cassette flanked by BsmBI sites on the substrate side, and a multiple cloning site flanked by BsaI sites on the enzyme side. The plasmid FRP793_insul-(lexA-box)₄-PminCYC1-Citrine-TCYC1 used to amplify the B-estradiol-inducible promoter, was a gift from Joerg Stelling (Addgene plasmid #58434) (Ottoz et al., 2014).

### EBY-Tune Strain Construction:

The MoClo-YTK plasmid kit was a gift from John Dueber (Addgene kit #1000000061). A receiver plasmid was constructed using Mo-Clo-YTK to integrate at the Leu2 marker of EBY100 under zeocin selection (LeuIV-ZeoR). The lexA-ER-haB112 transcription factor cassette was amplified from the plasmid FRP880_PACT1 (-1-520)-LexA-ER-haB112-TCYC1, a gift from Joerg Stelling (Addgene plasmid #58437; RRID: Addgene_58437). The transcription factor was cloned in LeuIV-ZeoR via BsaI assembly. The obtained plasmid LeuIV-ZeoR-LexA_TF was linearized with NotI and transformed into EBY100-kex2− by electroporation as described elsewhere (Benatuil et al., 2010).

Cassette assemblies: For module construction, 150 pmol of each the forward and reverse primers were mixed with 1 μL of T4 ligase buffer and 0.2 μL T4 polynucleotide kinase (T4 PNK) in 10 μL reaction. The oligos were phosphorylated and annealed using the following PCR program: 37° C. for 30 min, 98° C. for 5 min, 0.1° C./s ramp down to 4° C. Annealed modules were diluted 12.5-fold in water, kept on ice to be used immediately, or stored at −20° C.

To assemble modules in pYESS2, each module (0.5 μL) was mixed with 25 ng of pYESS2 in a 5 μL reaction containing 0.5 μL T4 ligase buffer, 0.25 μL BsmBI, 0.25 μL T7 ligase. Golden gate assembly program was as follows: (37° C. for 1 min, 16° C. for 2 min)×29 cycles, 37° C. for 60 min, 65° C. for 20 min, 4° C. Golden gate reactions were desalted by drop-dialysis (HVLP04700 Durapore, Millipore) and transformed in E. coli DH10B by electroporation, plated on LB+ampicillin (100 μg/mL) plates and incubated at 37° C. overnight. White clones were picked, miniprepped, and sequenced.

For cloning of the enzyme into pYESS2, the choice of ERS strength was encoded on the reverse primer used to amplify the enzyme gene. The enzyme was added to pYESS2 or pYESS2+substrate via a BsaI Golden Gate assembly. Routine transformations of pYESS2 plasmids in EBY100-Tune were performed in EZ-competent cells (Zymo Research, CA) and plated on YNB-CAA-glucose plates and incubated at 30° C.

### TEV Protease Library Construction and Sorting:

A TEV protease library with no ERS with a 1.5% error rate was prepared using Taq DNA polymerase. The combinatorial NNS site saturation library was prepared by overlap extension PCR. pYESS2 vectors were linearized by PstI and SphI, and yeast transformation was performed as described previously (Benatuil et al., 2010). Libraries were induced in YNB-CAA-Galactose supplemented with various concentrations of B-estradiol at a starting OD of 0.5. Typically, the number of induced cells corresponded to a ˜10-fold coverage of the initial library or the number of cells collected in the previous sorting round. For antibody labeling, cells were washed in cold PBS+0.5% BSA, followed by labeling in ice-cold PBS+0.5% BSA with anti-FLAG-PE (Biolegend, 0.25 μL/10⁷ cells) and anti-HA-FITC (Genscript, 0.5 μL/10⁷ cells) antibodies at a concentration of 10⁵ cells/μL and incubated on ice, in the dark, for 1 hour. Lastly, cells were washed in PBS+0.5% BSA and scanned or sorted by FACS. After sorting, the cells were plated on selective medium plates, and individual colonies were reanalyzed and confirmed by flow cytometry. The DNA was extracted from the confirmed yeast single colonies using a Zymoprep kit (Zymo Research, CA) and transformed into E. coli for Sanger sequencing.

### TEV Protease and MBP-ENLYFQS-GST (SEQ ID NO:16) Fusion Protein Expression and Purification:

TEV protease variants were cloned downstream of a maltose-binding protein and transformed into E. coli BL21-RIL as described previously (Tropea et al., 2009). TEV proteases were purified using Ni-NTA chromatography, followed by dialysis overnight in storage buffer (50 mM Tris-HCl, pH 8.0, 0.1 mM NaCl, 1 mM DTT, 10% glycerol). For kinetic analysis, freshly purified TEV proteases were used. The MBP (maltose binding protein) and the GST (glutathione-S-transferase) protein were fused with a peptide linker containing the TEV substrate ENLYFQS (SEQ ID NO:7), designated as MBP-ENLYFQS-GST (SEQ ID NO:16). The MBP-ENLYFQS-GST (SEQ ID NO: 16) fusion protein was expressed and purified on an amylose resin as described previously (Yi et al., 2013).

### Protease Characterization:

TEV kinetic assays were carried out as described previously with minor modification (Tropea et al., 2009). Kinetic assays were carried out in 50 mM Tris-HCl, pH 8.0 containing 1 mM EDTA, and 2 mM freshly-prepared DTT. In a 50 μL reaction, a total of 7.5 μM to 1 mM of substrate peptide (TENLYFQSGTRRW (SEQ ID NO: 33)) was incubated with 0.1-0.5 μM of purified enzyme at 30° C. for 10-30 min. Subsequently, the reactions were quenched with 10 μL of 5% trifluoroacetic acid (TFA), followed by HPLC analysis on a C-18 column using an acetonitrile gradient from 2% to 60% over 10 min at a flow rate of 0.62 mL/min. The product amount was calculated upon the integration area at 280 nm and converted to concentration using a product response curve (SGTRRW (SEQ ID NO: 34)). The MBP-ENLYFQS-GST (SEQ ID NO:16) fusion protein was used to monitor cleavage of proteins by TEV variants by SDS-PAGE analysis. The extent of cleavage of fusion proteins was calculated using the Image Lab Software (Bio Rad).

### Structural Modeling of TEV Variants:

The structure modeling was performed according to a previously published procedure (Fan et al., 2020). The wild-type TEV protease structure (PDB: 1LVB) and peptide substrate, ENLYFQS (SEQ ID NO:7), were derived from the protein data bank (Phan et al., 2002). The structure of eTEV was derived from simulation using the online ITASSER program (Yang et al., 2015). The modeling studies were performed using ZDOCK program (Chen et al., 2003), followed by subsequent refinement with the RDOCK protocol.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Arnau et al., Protein Expr Purif, 48 (1): p. 1-13, 2006.
Benatuil et al., Protein Eng Des Sel, 23 (4): p. 155-9, 2010.
Blommel and Fox, Protein Expression and Purification, 55 (1): 53-68, 2007.
Cabrita et al., Protein Sci. 16:2360-2367, 2007.
Chen et al., Proteins, 52 (1): p. 80-7, 2003.
Cherf and Cochran, Methods Mol Biol, 1319: p. 155-75, 2015.
Chou and Fasman, Biochemistry 13, 211, 1974.
Copeland et al., Nat Chem Biol, 12 (4): p. 254-60, 2016.
Craik et al., Biochem J, 435 (1): p. 1-16, 2011.
Daros et al., Journal of Virology; 73 (10): 8732-8740, 1999.
Davis et al., Escherichia coli. Biotechnology and Bioengineering; 65 (4): 382-388, 1999.
de Ruijter et al., Microb Cell Fact, 15: p. 87, 2016.
Engler and Marillonnet, Methods Mol Biol, 1116: p. 119-31, 2014.
Fan et al., ACS Chem Biol, 15 (1): p. 63-73, 2020.
Fang et al., Protein Expression and Purification; 51 (1): 102-109), 2007.
Fink et al., Nat Chem Biol,15 (2): p. 115-122, 2019.
Gai and Wittrup, Curr Opin Struct Biol, 17 (4): p. 467-73, 2007.
Gao et al., Science, 361 (6408): p. 1252-1258, 2018.
Giansanti et al., Nat Protoc, 11 (5): p. 993-1006, 2016.
Guan et al., Gene., 67 (1): 21-30, 1988.
Guerrero et al., Chem Biol, 11 (4): 961-70. 2016.
Huang et al., Proc Natl Acad Sci USA, 115 (47): p. E11025-E11032, 2018.
Kandror et al., EMBO Journal; 14 (23): 6021-6027, 1995.
Kapust and Waugh, Protein Expr. Purif. 19:312-318, 2000.
Kapust and Waugh, Protein Sci. 8:1668-1674, 1999.
Kapust et al., Biochem. Biophys. Res. Commun. 294:949-955, 2002a.
Kapust et al., Prot. Eng. 14:993-1000, 2001.
Kapust et al., Prot. Expr. Purif. 24:61-70, 2002b.
Kim et al., Elife, 8, 2019.
Konning and Kolmar, Microb Cell Fact, 17 (1): p. 32, 2018.
Kraft et al., Escherichia coli. Protein Expression and Purification; 52 (2): 478-484, 2007.
Kyte and Doolittle, J. Mol. Biol. 157:105-132, 1982.
LaVallie et al., Nature Biotechnology; 11 (2): 187-193, 1993.
Lee et al., ACS Synth Biol, 4 (9): p. 975-86, 2015.
Li et al., 12 (2): p. 510-518, 2017.
Li et al., FEBS Lett, 587 (8): p. 1155-63, 2013.
Lim et al., ACS Chem Biol, 12 (2): p. 336-341, 2017a.
Lim et al., Biotechnol J, 12 (5), 2017b.
Lin et al., Proc Natl Acad Sci USA, 105 (22): p. 7744-9, 2008.
Los G V, et al., ACS Chemical Biology. 3 (6): 373-82, 2008.
Marblestone et al., Protein Science; 5 (1): 182-189, 2006.
Mei et al., Front Bioeng Biotechnol, 7: p. 362, 2019.
Mei et al., J Biol Chem, 292 (50): p. 20707-20719, 2017a.
Mei et al., Microbiol Res, 196: p. 118-128, 2017b.
Nam et al., FEBS Open Bio, 10 (4): p. 619-626, 2020.
Needleman and Wunsch, J. Mol. Biol, 48:443-453, 1970.
Ottoz et al., Nucleic Acids Res, 42 (17): p. e130, 2014.
Packer et al., Nat Commun, 8 (1): p. 956, 2017.
Parks et al., Analytical Biochemistry; 216 (2): 413-417, 1994.
Pearson and Lipman, Proc. Natl. Acad. Sci. USA, 85:2444-2448, 1988.
Phan et al., J Biol Chem, 277 (52): p. 50564-72, 2002.
Pogson et al., Curr Opin Biotechnol, 20 (4): p. 390-7, 2009.
Pryor et al., Protein Expression and Purification, 10 (3): 309-319, 1997.
Sanchez and Ting, Nat Methods, 17 (2): p. 167-174, 2020.
Sandersjoo et al., Biotechnol J, 9 (1): p. 155-62, 2014.
Schulz et al., Principles in Protein Structure, Springer-Verlag, pp. 108-130, 1978.
Sellamuthu et al., PLOS One, 6 (7): p. e22554, 2011.
Smith et al., Gene.; 67 (1): 31-40), 1988.
Smith et al., J. Mol. Evol. 18:38-46, 1981.
Stein and Alexandrov, Proc Natl Acad Sci USA, 111(45): p. 15934-9, 2014.
Taft et al., ACS Chem Biol, 14 (9): p. 1888-1895, 2019.
Tropea et al., Methods Mol Biol, 498: p. 297-307., 2009
Van den Berg et al., J. Biotechnol. 121:291-298, 2006.
Varadarajan et al., Angew Chem Int Ed Engl, 47 (41): p. 7861-3, 2008a.
Varadarajan et al., Nat Chem Biol, 4 (5): p. 290-4, 2008b.
Varadarajan et al., Proc Natl Acad Sci USA, 102 (19): p. 6855-60, 2005.
Wang et al., Nat Biotechnol, 35 (9): p. 864-871, 2017.
Wu et al., J Biomed Biotechnol. Vol. 2009; 591923, 2009.
Wu et al., BioMed Research International, vol. 2009, Article ID 591923, 8 pages, 2009.
Yang et al., Nat Methods, 12 (1): p. 7-8, 2015.
Yi et al., Methods Mol Biol, 1319: p. 81-93, 2015.
Yi et al., Proc Natl Acad Sci USA, 110 (18): p. 7229-34, 2013.

1. A polypeptide comprising an active mutant TEV (Tobacco Etch Virus) protease, wherein the mutant TEV protease comprises:
(i) substitution mutations at amino acid residues corresponding to amino acids S3, P8, S31, and A231 of SEQ ID NO:1; and/or
(ii) a substitution mutation of arginine at amino acid residue corresponding to S219 of SEQ ID NO:1.

2. The polypeptide of claim 1, wherein the mutant TEV protease further comprises a substitution mutation at amino acid residue corresponding to amino acid T173 of SEQ ID NO:1.

3. The polypeptide of claim 1, wherein the active mutant TEV protease comprises alanine at position 173 (T173A).

4. The polypeptide of claim 1, wherein the mutant TEV protease comprises substitution mutations of: isoleucine at position 3 (S31), glutamine at position 8 (P8Q), threonine at position 31 (S31T), and a substitution mutation at position 231.

5. The polypeptide of claim 4 wherein, the mutant TEV protease comprises valine at position 231 (A231V).

6. (canceled)

7. The polypeptide of claim 4, wherein the mutant TEV protease comprises the substitution mutation of S219R.

8. The polypeptide of claim 4, wherein the mutant TEV protease comprises the substitution mutations of S3I, P8Q, S31T, T173A, and A231V.

9-11. (canceled)

12. The polypeptide of claim 8, wherein the mutant TEV protease comprises the substitution mutations of S3I, P8Q, S31T, T173A, S219R, and A231V.

13. The polypeptide of claim 1, wherein the polypeptide comprises or consists of SEQ ID NO:3, SEQ ID NO:4, or a sequence having at least 95% sequence identity thereto.

14-15. (canceled)

16. The polypeptide of claim 15, wherein the mutant TEV protease has an amino acid sequence at least 95% identical to (SEQ ID NO:1), and wherein the mutant TEV protease comprises substitution mutations at the amino acid residues corresponding to positions S3I, P8Q, S31T, T173A, and A231V of (SEQ ID NO:1).

17-18. (canceled)

19. The polypeptide of claim 1, wherein the polypeptide can cleave Glu-X-X-Tyr-X-Gln/Xa, (SEQ ID NO:5) at a rate that is faster than the wild-type TEV protease.

20. The polypeptide of claim 19, wherein the polypeptide can cleave ENLYFQG (SEQ ID NO:6) or ENLYFQS (SEQ ID NO:7) at a rate of greater than a k_{cat} value of 0.17 s⁻¹.

21-23. (canceled)

24. The polypeptide of claim 1, wherein the polypeptide further comprises a polypeptide tag sequence.

25. The polypeptide of claim 24, wherein the polypeptide tag sequence is a metal binding tag, a histidine tag or polyhistidine tag (His-tag), a HQ tag, a glutathione S-transferase (GST) tag, a N-Utilization substance (NusA) tag, thioredoxin (TRX), trigger factor, SUMO, or a polyarginine tag.

26. The polypeptide of claim 1, wherein the polypeptide is covalently bound to a maltose binding protein (MBP).

27-28. (canceled)

29. The polypeptide of claim 1, wherein the polypeptide comprises a polyhistidine tag or a polyarginine tag.

30-31. (canceled)

32. The polypeptide of claim 29, wherein the polypeptide comprises an N-terminal polyhistidine tag and a C-terminal polyarginine tag.

33-36. (canceled)

37. The polypeptide of claim 1, wherein the polypeptide is covalently attached to or expressed as a fusion protein with a fluorescent protein.

38-39. (canceled)

40. The polypeptide of claim 1, wherein the mutant TEV protease comprises SEQ ID NO:3, SEQ ID NO:4, or SEQ ID NO:18.

41. A method of cleaving an amino acid bond in a polypeptide comprising contacting the polypeptide with a mutant protease of claim 1.

42-50. (canceled)

51. A kit comprising the polypeptide of claim 1 and a suitable container.

52-54. (canceled)


SEQUENCE LISTING (34 sequences)

SEQ ID NO: 1 - protein, 234 aa, Artificial sequence
  Synthetic amino acid
SLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMSKPEEPFQPVKEATQLMN

SEQ ID NO: 2 - protein, 239 aa, Artificial sequence
  Synthetic amino acid
SLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMSKPEEPFQPVKEATQLMNRRRRR

SEQ ID NO: 3 - protein, 238 aa, Artificial sequence
  Synthetic amino acid
ILFKGQRDYNPISSTICHLTNESDGHTTTLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFANTNNYFTSVPKNFMELLTNQEAQQWVSGWRLNADSVLWGGHKVFMRKPEEPFQPVKEVTQLMRRRRR

SEQ ID NO: 4 - protein, 238 aa, Artificial sequence; identical to SEQ ID NO: 3
  Synthetic amino acid

SEQ ID NO: 5 - protein, 7 aa, Artificial sequence
  Synthetic amino acid
  misc_feature (2)..(3): Xaa can be any naturally occurring amino acid
  misc_feature (5)..(5): Xaa can be any naturally occurring amino acid
  misc_feature (7)..(7): Xaa can be any naturally occurring amino acid
EXXYXQX

SEQ ID NO: 6 - protein, 7 aa, Artificial sequence
  Synthetic amino acid
ENLYFQG

SEQ ID NO: 7 - protein, 7 aa, Artificial sequence
  Synthetic amino acid
ENLYFQS

SEQ ID NO: 8 - protein, 5 aa, Artificial sequence
  Synthetic amino acid
RRRRR

SEQ ID NO: 9 - protein, 297 aa, Artificial sequence
  Synthetic amino acid
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 10 - protein, 24 aa, Artificial sequence
  Synthetic amino acid
HENLYFQSFLAGENLYFHSHAERS

SEQ ID NO: 11 - protein, 63 aa, Artificial sequence
  Synthetic amino acid
QLLEGRYHHHHHHGGGGSENLYFQSGSDYKDDDDKGSENLYFESGSYPYDVPDYAGSFEHDEL

SEQ ID NO: 12 - DNA, 202 bp, Artificial sequence
  Synthetic nucleic acid
atgcagttacttgaaggtagatcacatcaccatcaccatcacggtggaggaggctcagagaacctttatttccaaagcggcagtgattataaagatgacgacgataaaggttcggagaatctatattttgaaagtggaagctacccatacgacgttccagactacgctggtagtttcgaacacgacgaattgtagtaacaat

SEQ ID NO: 13 - protein, 58 aa, Artificial sequence
  Synthetic amino acid
RYHHHHHHGGGGSENLYFQSGSDYKDDDDKGSENLYFESGSYPYDVPDYAGSFEHDEL

SEQ ID NO: 14 - protein, 13 aa, Artificial sequence
  Synthetic amino acid
TENLYFQSGGTRW

SEQ ID NO: 15 - protein, 13 aa, Artificial sequence
  Synthetic amino acid
TENLYFQSTRGGW

SEQ ID NO: 16 - protein, 13 aa, Artificial sequence
  Synthetic amino acid
MBPENLYFQSGST

SEQ ID NO: 17 - protein, 234 aa, Artificial sequence
  Synthetic amino acid
GESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMSKPEEPFQPVKEATQLM

SEQ ID NO: 18 - protein, 234 aa, Artificial sequence
  Synthetic amino acid
GEILFKGQRDYNPISSTICHLTNESDGHTTTLYGIGFGPFIITNKHLFRRNNGTLLVQSLHGVFKVKNTTTLQQHLIDGRDMIIIRMPKDFPPFPQKLKFREPQREERICLVTNFQTKSMSSMVSDTSCTFPSSDGIFWKHWIQTKDGQCGSPLVSTRDGFIVGIHSASNFANTNNYFTSVPKNFMELLTNQEAQQWVSGWRLNADSVLWGGHKVFMRKPEEPFQPVKEVTQLM

SEQ ID NO: 19 - protein, 234 aa, Artificial sequence
  Synthetic amino acid
GESLFKGPRDYNPISSTICHLTNESDGHTTSLYGIGFGPFIITNKHLFRRNNGTLLVQSLHGVFKVKNTTTLQQHLIDERDMIIIRMPKDFPPFPQKLKFREPQREERICLVTNFQTKSMSSMVSDTSCTFPSSDGIFWKHWIQTKDGQCGSPLVSTRDGFIVGIHSASNFANTNNYFTSVPKNFMELLTNQEAQQWVSGWRLNADSVLWGGHKVFMVKPEEPFQPVKEATQLM

SEQ ID NO: 20 - DNA, 36 bp, Artificial sequence
  Synthetic nucleic acid
agataccaccatcatcaccatacacggtggaggagg

SEQ ID NO: 21 - DNA, 35 bp, Artificial sequence
  Synthetic nucleic acid
tgagcctcctccaccgtgatggtgatgatggtggt

SEQ ID NO: 22 - DNA, 25 bp, Artificial sequence
  Synthetic nucleic acid
ctcagagaacctttattccaaagcg

SEQ ID NO: 23 - DNA, 26 bp, Artificial sequence
  Synthetic nucleic acid
ctgccgctttggaaataaaggttctc

SEQ ID NO: 24 - DNA, 29 bp, Artificial sequence
  Synthetic nucleic acid
gcagtgattataaagatgacgacgataaa

SEQ ID NO: 25 - DNA, 29 bp, Artificial sequence
  Synthetic nucleic acid
aacctttatcgtcgtcatctttataatca

SEQ ID NO: 26 - DNA, 28 bp, Artificial sequence
  Synthetic nucleic acid
ggttcgagaatctatattttgaaagtgg

SEQ ID NO: 27 - DNA, 29 bp, Artificial sequence
  Synthetic nucleic acid
gcttccactttcaaaatatagattctccg

SEQ ID NO: 28 - DNA, 33 bp, Artificial sequence
  Synthetic nucleic acid
aacgtacccatacgacgttccagactacgctgg

SEQ ID NO: 29 - DNA, 33 bp, Artificial sequence
  Synthetic nucleic acid
actaccagcgtagtctggaacgtcgtatgggta

SEQ ID NO: 30 - DNA, 28 bp, Artificial sequence
  Synthetic nucleic acid
tagtttcgaacacgacgaattgtagtaa

SEQ ID NO: 31 - DNA, 28 bp, Artificial sequence
  Synthetic nucleic acid
attgttactacaattcgtcgtgttcgaa

SEQ ID NO: 32 - protein, 19 aa, Artificial sequence
  Synthetic amino acid
AGAAGAFLAGENLYFQSHA

SEQ ID NO: 33 - protein, 13 aa, Artificial sequence
  Synthetic amino acid
TENLYFQSGTRRW

SEQ ID NO: 34 - protein, 6 aa, Artificial sequence
  Synthetic amino acid
SGTRRW